(19) United States
(12) Patent Application Publication
(10) Pub. No.: US 2025/0197795 A1
(43) Pub. Date: Jun. 19, 2025

(54) MICROBIAL EXTRACTS, USES AND APPLICATIONS

(71) Applicant: FUMI Holding B.V., Wageningen (NL)

(72) Inventors: Edgar Suarez GARCIA, Wageningen (NL); Corjan VANDENBERG, Wageningen (NL)

(21) Appl. No.: 18/849,281

(22) PCT Filed: Mar. 22, 2023
(86) PCT No.: PCT/EP23/57331

§ 371 Date: Sep. 20, 2024

(30) Foreign Application Priority Data
Mar. 23, 2022 (NL) 2031387

(51) Int. Cl. C12N 1/06; A23J 1/18; A23J 3/20; A23L 33/145; C12R 1/84; C12R 1/85

(52) U.S. Cl. CPC C12N 1/063; A23J 1/18; A23J 3/20; A23L 33/145; C12N 1/066; C12R 2001/84; C12R 2001/85

(57) ABSTRACT

The invention relates to a method for producing a microbial cell extract with improved functional properties. The invention further relates to a microbial cell extract obtained by or obtainable by said method. The invention further relates to the use of said microbial cell extract with improved functionality, with applications in gelation agents, thickening agents, foaming agents, emulsification agents, texturing agents and other suitable applications.

### TECHNICAL FIELD

The invention relates to a method for improving the functionality of microbial cell extracts. The invention further relates to a microbial cell extract with improved functionality obtained by or obtainable by said method. The invention further relates to the use of said microbial cell extract with improved functionality, with applications in gelation agents, thickening agents, foaming agents, emulsification agents, texturing agents and other suitable applications.

### BACKGROUND OF THE INVENTION

Microorganisms have played an essential role in the food industry, not only as bioconversion agents in traditional foods and beverages such as cheese, beer, wine, tofu and tempeh, but also as sources of biomass, nutritional ingredients and functional ingredients. More recently, technological developments have led to the advancement of cultured meat products, in which animal cells are cultivated and grown in bioreactors to mimic muscle tissue of animals.

The most common example of direct usage of microorganisms in the form of a microbial biomass in foods is the so-called single cell protein (SCP), particularly in the 1940s, when the production of bacterial, fungal and yeast biomass was accelerated in order to close the protein gap and to supply enough food worldwide. In addition to SCP, microorganisms can be used for the targeted production of functional proteins with high market value. Examples of this are the expression of milk and egg proteins in host organisms for the large-scale production of animal-free dairy and egg-containing ingredients.

Moreover, microorganisms can be processed to purify certain components of interest, or to extract target compounds present intracellularly, in the cell walls or membranes or in specialized organelles. Examples of such compounds are lipids and pigments from algae (Silva et al., 2020) polysaccharides from macroalgae, or proteins from yeast (Kinsella and Shetty, 1978).

For the specific case of yeast and yeast derived proteins, there is a vast range of products developed at commercial scale, of which most notable are yeast extracts and yeast proteins. The production of yeast extracts and yeast proteins have been traditionally done with a series of processes that involve cell lysis, separation and protein purification (U.S. Pat. No. 3,888,839A and EP3670646A1). The most common methods for cell lysis are autolysis and plasmolysis, in which the intrinsic biochemical reactions are responsible for the disintegration of the cell wall. After this, a separation step is implemented in order to produce a soluble fraction and an insoluble fraction. Methods often implemented for this step are centrifugation and filtration. Furthermore, protein purification is conducted with methods such as isoelectric precipitation (Kinsella and Shetty, 1978). Due to the high temperatures and long exposure times, extreme variations of pH, and the usage of salts or solvents, the traditional processing of yeast results in protein denaturation and hydrolysis. Even if the final protein fractions have high purity, the functional properties are limited (Kinsella and Shetty, 1978). Functional properties, such as foaming, emulsification and gelation, are essential for technical applications in many food applications that include bakery, confectionary, meat analogues and dairy analogues.

Vananuvat and Kinsella (1975) and Kinsella and Shetty (1978) provide a complete overview of the nutritional and functional properties of proteins from microbial biomass, in particular proteins from yeast. The authors clearly indicate that under the traditional extraction methods, the functional properties of yeast proteins remain limited. Moreover, the authors present methods to improve their functionality, mostly via protein functionalization with chemical reactions.

U.S. Pat. No. 3,887,431 presents a method to produce a soluble protein isolate from yeast which displays technical functionality, including gelation. Similarly, U.S. Pat. No. 3,888,839 describes a method for producing a yeast protein that can be used as meat extender. GB1578235A also claims a process for making yeast proteins with similar functionality similar to egg proteins, but no further information on the functional properties of these proteins or on methods to improve functional properties are disclosed. U.S. Pat. No. 3,867,554 and U.S. Pat. No. 5,756,135 describe methods for preparing yeast glycan/solid extracts which can be used as fat replacers. U.S. Pat. No. 10,407,600B2 and U.S. Pat. No. 2,603,630A present methods for making yeast protein extracts with adhesive properties. WO2006067145A1 and WO2018002505A1 claim methods for producing yeast protein extracts and their application for the stabilization of wines, and for the control of haziness in beer. EP3670646A1 describes an ultra-filtration method to purify soluble yeast proteins, such proteins displaying thermal gelation properties.

More recently, a novel process has been reported in which soluble yeast proteins are purified using ultrafiltration (WO2020127951A2), yielding a protein concentrate which exhibits gel-like properties. The patent application PCT/EP2021/075137 presents a method to produce aqueous microbial extracts rich in large and small fragments, which display distinct functional properties.

Despite the various scientific reports and inventions related to microbial proteins (particularly yeast proteins), and the numerous claims regarding functional properties and wide range of applications, microbial proteins have not been clearly explored in terms of their performance as foaming, emulsification and gelation agents, and no novel methods have been presented to improve their functionality, rather than just chemical functionalization strategies extensively known in the art.

There is therefore a need to develop methods to improve the functional properties of microbial cell extracts, particularly in relation to foaming, emulsification and gelation. The present invention addresses this need.

### SUMMARY OF THE INVENTION

Described herein is a method of preparing a microbial cell extract, said method comprising: providing a microbial biomass; subjecting said microbial biomass to disintegration; subjecting the resulting disrupted biomass to solid-liquid classification to obtain a light fraction and a heavy fraction. Said method may also subsequently optionally comprise subjecting the heavy fraction and/or light fraction and/or a combination of the light and heavy fraction to a purification step, and/or concentration step, and/or preparation of an alkaline suspension, and/or further solid-liquid classification, and/or emulsification, and/or dilution and/or pH adjustment, and/or stirring, and/or drying and/or thermal treatment.

In an aspect of the invention, there is provided a method of preparing a microbial cell extract, the method comprising:
a) providing a microbial biomass in an aqueous alkaline suspension, at pH 7-11;
b) mechanically disintegrating the microbial biomass at a temperature below 40 deg C using a non-denaturing process, such that the disintegrated biomass consists of a population of soluble compounds and suspended fragments characterized by a bimodal distribution and a particle size distribution (psd) at an average of around D50<4.5 μm;
c) subjecting the disintegrated biomass to a solid-liquid separation process, to separate the disintegrated biomass into a light fraction (also referred to herein as an extract rich in small fragments) and a heavy fraction (also referred to herein as an extract rich in large fragments), wherein the light fraction consists of a population of soluble compounds and suspended fragments characterized by a bimodal distribution and a psd of D50 around 0.5 μm or lower; and the heavy fraction consists of a population of soluble compounds and suspended fragments having a psd of D50>0.5 μm; and
d) further processing either or both of the light fraction and heavy fraction to optimize one or more functional properties of said fraction(s).

In some embodiments, the heavy fraction consists of a population of soluble compounds and suspended fragments having a psd of D50>4 μm.

In an embodiment any combination of the fractions obtained as described above may be combined in any proportion depending on the specific application of the microbial cell extract.

As will be described herein, the one or more functional properties may be selected from gelation properties, foaming properties, emulsification properties, texture properties, glazing properties, and/or browning properties. The further processing of step d) is a non-chemical further processing step; and preferably does not alter the chemical structure of the components of the fraction(s). Shetty and Kinsella (1978) and Vananuvat and Kinsella (1975) have described in detail the functional properties of proteins from yeast. These authors highlight the limited functionality of yeast proteins, in particular foaming, and show that the functional properties can be improved by means of protein functionalization, for example succinylation and phosphorylation. One of the key aspects of the present invention is that methods are provided to enhance the functionality of light and/or heavy fraction, without the need of bio-chemical reactions or complex purification methods. In general, different processing steps described herein may be combined when processing a given fraction, unless otherwise noted.

The functional properties described herein are primarily desirable functional properties for use in food manufacturing (including animal feed), although it will be appreciated that such functional properties including gelation, emulsification and/or foaming may also be desirable for other applications, such as cosmetic production.

Microbial biomass, which comprises microbial cells, has been used traditionally to produce a broad range of products of industrial interest, or has been used directly in a number of applications. Most industrial or commercial applications make use of a selected group of microbial biomass strains from the domains bacteria, yeast, fungi and algae. In overall terms, products obtained from microbial biomass are either intracellular or extracellular. Extracellular products are excreted by the cells into the bulk medium, usually an aqueous phase. Intracellular products, on the contrary, remain inside of the cells. In order to obtain intracellular products, additional processing is needed to release these products from the cells (by breaking the cell membrane or wall) and to further separate the compounds of interest from the remaining biomass and other impurities.

In the particular field of food applications, microbial biomasses have been used as a source of proteins (single cell protein-SCP), as nutritional supplements, or to produce various ingredients and additives.

Microbial biomasses are often used in the form of extracts, for which the microbial cells forming said biomass need to be disrupted/disintegrated. Extracts prepared from several different starting materials are known, such as fungal extract, algae extract and yeast extract. Of these extracts, the most commonly used is that derived from yeast, the so-called yeast extracts (hereinafter referred to as “YE”). YE are (and can be) applied in a broad range of products ranging from growth media for culturing cells for laboratories to nutritional supplements and flavour enhancers for the food industry. Production processes of YE are well known. In general, yeast cells, mostly from the genus Saccharomyces, are disrupted (=disintegrated) by means of heat induced or chemically induced autolysis (or plasmolysis), followed by a step of incubation at high temperatures (>50° C.) in order to activate endogenous enzymes, which break down (=digest) the large intracellular products such as proteins and nucleic acids into smaller components thereof such as peptides, amino acids and nucleotides. The digested slurry that is obtained is then further purified and supplemented-depending on the final application—to provide an extract that can be commercialized as YE.

The microbial biomass used in the present invention can be obtained from several microbial types, including microalgae, yeast, bacteria and fungi. Examples of genus from which the microbial biomass may be derived for the microbial extract may be produced are Saccharomyces and Pichia (yeast), Tetraselmis, Chlorella, Arthrospira (algae), Fusarium (fungi), Methylobacterium (bacteria) and Lactobacillus (bacteria). Preferably the microbial biomass is derived from yeast, more preferably from the genus Saccharomyces and/or Pichia. Yeasts which may be used in the present invention include Saccharomyces, such as S. cerevisiae, S. chevalieri, S. boulardii, S. bayanus, S. italicus, S. delbrueckii, S. rosei, S. micro-ellipsodes, S. carlsbergensis, S. bisporus, S. fermentati, S. pastorianis, S. rouxii, or S. uvarum; a yeast belonging to the genus Schizo-saccharomyces, such as S. japonicus, S. kambucha, S. octo-sporus, or S. pombe; a yeast belonging to the genus Hansenula, such as H. wingei, H. arni, H. henricii, H. americana, H. canadiensis, H. capsulata, or H. polymorpha; a yeast belonging to the genus Candida, such as C. albicans, C. utilis, C. boidinii, C. stellatoidea, C. famata, C. tropicalis, C. glabrata, or C. parapsilosis; a yeast belonging to the genus Pichia, such as P. pastoris, P. kluyveri, P. polymorpha, P. barkeri, P. cactophila, P. rhodanensis, P. cecembensis, P. cephalocereana, P. eremophilia, P. fermentans, or P. kudriavzevii; a yeast belonging to the genus Kluyveromyces, such as K. marxianus; and a yeast belonging to the genus Torulopsis, such as T. bovina, or T. glabrata.

In a preferred embodiment the microbial biomass is free from polluting material—for example, the biomass may be purified by centrifugation followed by washing and resuspension; several rounds of washing and resuspension may be used. In another preferred embodiment said microbial biomass is prepared in an aqueous alkaline suspension in the pH range of 7-11, more preferably pH˜9, optionally said microbial biomass is at a concentration of around 50-150 g/L in the aqueous suspension, preferably ˜100 g/L.

Generally, cell disintegration methods can be classified as being either non-mechanical or mechanical. Non-mechanical disintegration methods can be further sub classified into three categories: physical disintegration (e.g. by means of decompression, osmotic shock, thermolysis, ultrasonics, or freezing and thawing), chemical disintegration (e.g. by use of solvents, detergents, chaotropes, acids and bases, or chelates) and enzymatic disintegration (e.g. by autolysis, phage lysis, or lytic enzymes). The present invention is preferably related to mechanical disintegration methods. Examples of mechanical disintegration methods are ball mills, including bead mills, and homogenizers.

Ball mills (including bead mills) can be either vertical and horizontal and use a grinding medium which is present in the grinding chamber. A motor drives a rotor to rotate the cell suspension at a high speed. The cell suspension and the grinding material (e.g.

beads) generate shearing force to break the cells. This results in the release of intracellular materials into the aqueous suspension and will also result in cell fragmentation (i.e., disintegration). With increasing rotor speed, the shear force increases and the cell breakage increases. With decreasing grinding material size, the cell breakage usually increases. Other parameters affect the performance of the disintegration process. The skilled person is capable of selecting the right parameters and variables in accordance to the present invention.

Homogenizers work under high-pressure and are in fact a positive-displacement pump that forces a cell suspension through a valve, before impacting the stream at high velocity on an impact ring. Often, several passes at high-pressure are required, which may lead to rising temperatures causing local denaturation of labile molecules.

Preferably the mechanical disintegration step is performed using bead milling or high pressure homogenization. Most preferably the mechanical disintegration step is performed using bead milling. Preferably, the disintegration step is carried out at a pH in the range of 7-11 (optionally 8-10, or 8.5-9.5 or most preferably 9), and at a temperature in the range of approximately 10-30° C., more preferably approximately 15-25° C., even more preferably 20-25° C., and most preferably around 25° C. Carrying out the disintegration step within the said pH and temperature ranges has the technical effect of preventing the denaturation of proteins and other labile molecules (that is, it is a non-denaturing process) and consequently preventing the activation of lytic enzymes, proteases or other hydrolytic enzymes present in the microbial biomass. The skilled person is able to adjust the process parameters of the disintegration method (speeds, flows, filling ratios, bead sizes, pressures, etc) in order to keep the temperature preferably at <25° C. and to reach the desired psd target.

The disintegration step results in the production of a population of soluble compounds and suspended fragments characterized by a bimodal distribution and a particle size distribution (psd) at an average of around D50<4.5 μm. It is recognized that the skilled person would be able to adjust the parameters used during the disintegration step accordingly to achieve the desired targets of their application. During the course of the cell disintegration process, the psd varies, showing a decrease in the peak of intact cells, and a consequent increase in the peak of cell fragments. The disintegration process is run until a specific psd is obtained. For the example case of yeast, the specific surface area increases over time, while the D[3,2], D[4,3], D10, D50, D90 decrease over time. Other psd parameters may also be considered; for example, for yeast, one desired target psd may be D10<0.5 μm, D50<4.5 μm, D90<7.5 μm and D[3,2]<2 μm, D[4,3]<4.5 μm.

In the art, particle size distribution is reported as a volume distribution. Taking D50 as an example it is known in the art that this can also be referred to as Dv50; the terms D50 and Dv50 are used interchangeably herein. The D50 or Dv50 is defined in the art as the maximum particle size, measured by diameter, below which 50% of the sample volume exists, also known as the median particle size (diameter) by volume. This concept is illustrated in FIG. 15. Numerous analytical techniques and approaches exist for particle size analysis.

A particle size analyser is an analytical instrument that measures, visualises, and reports a particle size distribution for a given particle or droplet population. Laser diffraction particle size analysers calculate particle size from the angle of light scattered by a stream of particles passing through a laser beam. This technique allows for continuous measurement of bulk material across a wide size range. The size limits and sensitivity of a laser diffraction particle analyser depend on the number and placement of detectors in the instrument. Dynamic light scattering particle analysers are mainly used for analysing particles in solution. Dynamic light scattering determines size from the fluctuations in scattered laser light intensity created by the particles' Brownian motion. Induced grating particle size analysers identify the size of small particles in solution by electrically aligning the particles and then measuring their diffusion.

In the context of the present invention the D50 was determined using a laser diffraction particle size analyser, the Malvern Mastersizer 2000, with a dispersant RI of 1.33 and a particle/material RI of 1.34, using a general-purpose analysis Model MS2000, and the Mie scattering model. Disintegration methods are used to obtain a bimodal distribution and a particle size distribution (psd) at an average of around D50<4.5 μm as described above. Examples of cell disintegration methods according to the prior art are the following. U.S. Pat. No. 3,888,839A discloses a process for obtaining a protein isolate from yeast cells, wherein the yeast cells are ruptured by high-pressure homogenization (mechanical disintegration) and subsequent incubation. EP1199353A1 discloses a process for producing yeast extracts by treating yeast suspensions or yeast pastes and separating off the insoluble constituents, in which the yeast suspensions or yeast pastes are subjected to high-voltage electrical pulses (physical disintegration). EP2774993A1A discloses the use of a cell wall-decomposing enzyme (enzymatic disintegration) that does not contain protease and then heat-treating the product for 10 to 20 minutes at 70-80° C.

Microbial cells present in microbial biomass suspensions contain mostly proteins, carbohydrates, lipids and minerals. Proteins and other labile molecules experience unfolding, denaturation and degradation when exposed to high temperatures, long incubation times, extreme values of pH, solvents, salts and other harsh chemicals. When proteins and other functional molecules are denatured (tertiary and quaternary structure is lost), (part of) their functional activity is lost. Upon denaturation (unfolding), proteins lose their ability to interact with hydrophilic and hydrophobic surfaces, and also their ability to rearrange and form network-like structures upon heat-cooling treatments is affected. The present inventors have observed that the use of mechanical disintegration at the conditions described herein (for example, at the stated pH range) is sufficiently gentle to prevent unfolding, denaturation and/or degradation of proteins and other labile molecules, and therefore, necessary to preserve the functional properties, in particular gelation behaviour, water holding capacity and oil holding capacity.

The aqueous suspension comprising microbial biomass may further comprise cytoplasmic material or other extracellular material produced during propagation or fermentation.

In an embodiment of the method according to the present invention, the microbial biomass comprises microbial cells selected from unicellular or colonial prokaryotes and eukaryotes and one or more combinations thereof. In a preferred embodiment, the microbial cells are selected from the group consisting of yeast, algae, bacteria, fungi, and one or more combinations thereof. In a specific embodiment, the microbial cells are yeast.

Bead sizes that may be considered are in the range of 0.1-5 mm, preferably in the range of 0.5-1 mm. Suitable bead materials include, but are not limited to, zirconium and glass. Bead fillings (the percentage of the bead mill chamber that is filled with beads) that may be considered suitable are in the range of 40-90%, preferably in the range of 65-80%, more preferably 75%, based on the total available volume of the bead mill chamber.

Rotational speeds that may be considered suitable are in the range of 1-20 m/s. Depending on the configuration and geometry of each bead mill, the skilled person can estimate the corresponding rotor speeds in rpm. Suitable rotational speeds in rpm are for example 500-5000 rpm, preferentially 1000-3000 rpm.

Concentration of microbial cells that may be considered suitable are in the range of 2-25% dry weight.

In one specific embodiment, microbial cells are disintegrated using a Dyno-mill Research Lab (WAB) bead mill. Cells can also be disrupted by shear forces, such as with the use of blending (such as with a high speed or Waring blender as examples), the French press, or even centrifugation in case of weak cell walls, to disintegrate cells.

In an embodiment, cell disintegration takes place without the addition of chemicals and/or solvents.

The disintegration step preferably results in the production of a population of soluble compounds and suspended fragments characterized by a bimodal distribution and a particle size distribution (psd) at an average of around D50<4.5 μm. It is recognized that the skilled person would be able to adjust the parameters used during the disintegration step accordingly to achieve the desired targets of their application.

In a preferred embodiment the solid-liquid separation step is selected from a method known in the art including, but not limited to, centrifugation, decantation and filtration. More preferably, separation is performed by centrifugation, in a most preferred embodiment, separation is performed using centrifugation with a mild centrifugal field. An example of a mild centrifugal field is separation for <15 min at <4000 rcf in a bench top centrifuge. In an embodiment, said separation step is centrifuging at a centrifugal force equal to or smaller than 4000 relative centrifugal force (rfc). In an embodiment, said centrifuging takes place for a period of time equal to or shorter than 20 min. In a specific embodiment, said centrifuging takes place for a period of time equal to or shorter than 15 minutes. In another specific embodiment centrifuging takes place at ˜15° C. It will be understood that the skilled person is able to adjust the centrifugation parameters, depending on the type of equipment used, to reach the target characteristics (for example, psd) in both extracts.

The solid-liquid separation step results in the production of a light fraction (extract rich in small fragments) and a heavy fraction (extract rich in large fragments), as described in the patent application PCT/EP2021/075137.

In one embodiment the separation step of the method yields a light fraction comprising small cell fragments in the range of around 0.1-3 μm and a heavy fraction comprising large cell fragments that are at a size of >1 μm.

Preferably separation yields a volumetric ratio wherein the light phase has a maximum volumetric ratio of 0.1, more preferably <0.05 to total starting fraction. Preferably the heavy phase has a volumetric ratio of heavy fraction to total starting fraction of at least >0.65, more preferably >0.9. An estimate of the volumetric ratio obtained from separation is set out in Example 1. The skilled person would be able to adjust the separation parameters, for example, centrifugation parameters such as but not limited to any number of, time, g force, and/or sigma factor. It would be recognised by the skilled person that how said parameters are adjusted would vary depending on the centrifugation unit employed to achieve the desired classification target.

In an embodiment of the invention, the light and/or heavy fraction is further processed by purification and/or concentration and/or pH adjustment, and/or by emulsification methods. Examples of concentration methods include but are not limited to filtration, evaporation, freeze concentration and pervaporation. Examples of purification methods include but are not limited to isoelectric precipitation, coagulation, adsorption and filtration. It is known in the art that filtration methods, such as reverse osmosis (RO), nanofiltration (NF), ultrafiltration (UF) and microfiltration (MF) have been used to concentrate and purify proteins from yeast. Examples of such developments are given by Levesley at al., (2000), Shetty and Kinsella (1978) and Kollar et al., (1992).

In a preferred embodiment the light fraction is purified and/or concentrated so that said processed light fraction has a dry weight (DW) content of at least 10%, preferably at least 20% and even more preferably at least 30%. The processed light fraction preferably has a dry weight (DW) content of 10-30%, 20-30%, or around 30%.

In a preferred embodiment the light fraction is concentrated using filtration. More preferably the light fraction is concentrated using diafiltration. Diafiltration is a process for separation and purification of the target product out of the main solution containing the other small molecular weight (MW) substances, e.g. salts, sugars and amino acids. The term ‘diafiltration’ means a combination of ‘dilution’ and ‘filtration’. A buffer solution or demineralized water is added into the concentrate or retentate to make up the lost permeate water during filtration, in such a way keeping the concentration of rejected compounds (i.e. target products) constant, while diluting the unwanted small MW compounds for their gradual ‘washing out’ through filtration cycles. Preferably the light fraction is concentrated using dialfiltration where the ratio of water to the light fraction is approximately 1:1 and wherein preferably the membrane used for diafiltration is in the range of 10 kDa-0.2 μm, more preferably 100 kDa-1000 kDa.

In an embodiment purifying and/or concentrating the light fraction as described above is carried out to retain fragments <0.3 μm and large molecular weight compounds of >100 kDa.

The light fraction subjected to purification and/or concentration and/or pH adjustment and/or emulsification, as described above is referred to hereafter as the processed light fraction.

The present inventors have determined that the processed light fraction has foaming and/or emulsification and/or heat-set gelation properties that are improved compared to the unprocessed light fraction. Preferably, said improved heat set gelation properties of the processed light fraction are stable for at least 5 days without the addition of stabilisers and/or other preservatives when said processed light fraction is stored preferably at a temperature of ˜4° C. and preferably in the dark. The processed light fraction exhibits its maximum heat-set gelation capacity when applied as a wet ingredient; when the processed light fraction is used as a wet ingredient said processed light fraction has a DW content>5%, preferably a DW up to about 10%, 15%, 20%, 25%, 30%, 35%, 40% is considered “wet”.

The method may further comprise the step of drying the processed light fraction or heavy fraction to remove moisture; preferably the drying step is carried out so as to reach a moisture content of <15%, <10% or preferably <5%. In an embodiment, drying the light fraction from a moisture content of at least 85%, to a lower moisture content of <15% enhances functionality of the processed light fraction.

The method may further comprise the step of adjusting the pH of the processed light fraction to a pH in the range of 5-8, more preferably pH 6-7. These pH ranges provide superior heat set gelation performance.

The processing step may comprise preparing an emulsion of the fraction; this may be of the heavy fraction, of the light fraction, or (preferably) of the processed light fraction which has previously been concentrated and/or purified as described herein. Alternatively, the emulsion is of a combination of the light fraction and/or heavy fraction and/or processed light fraction. Preferably the emulsion comprises a suspension of the fraction in a mixture oil:water at a mass ratio of at least 0.25 and less than 2.5. More preferably, the ratio oil to water is around 1. In a preferred embodiment the fraction is dispersed first in the oil phase, followed by the addition of the water phase, with sufficient shear to ensure a homogenous dispersion. Emulsion systems created under this method result in superior gelation hardness, either as single ingredient or incorporated as functional ingredient in food products such as meat analogues and cheese analogues. What is considered “sufficient shear” may depend on the type of equipment used to prepare the emulsion, but can be readily determined by the skilled person. As an example, emulsions may be prepared at lab scale using a rotor-stator homogenizer, the mixing intensity adjusted, and the corresponding droplet size of the oil phase dispersed in water measured. If, as an example, the average droplet size of the oil droplets in the emulsions does not vary significantly from 40 to 65 rpm, then it would be considered that the shear caused by the mixer at 40 rpm is sufficient to prepare the emulsion.

In an embodiment the ratio of oil:water of the emulsion is at least 0.25, preferably around 1, preferably also less than 2, and less than 1.5. A ratio of oil:water of at least 0.25 gives better heat-set gelling properties including at least one of increased gel hardness, excellent gel texture and higher juiciness. Further, an emulsion with a ratio of oil: water of at least 0.25 reduces the sensory intensity of the concentrated light fraction.

In an embodiment the ratio of oil:water of the emulsion is >2.5, and preferably less than 5, less than 4, less than 3. This provides a more porous and/or stiffer texture of a heat-set gel obtained compared with lower ratios.

The processing step may further comprise adjusting the pH of the fraction to an alkaline pH; this may be of the heavy fraction, of the light fraction, or (preferably) of the processed light fraction which has previously been concentrated and/or purified as described herein. Alternatively, this may be a combination of the heavy fraction and/or light fraction and/or processed light fraction. In one embodiment, adjusting the pH of at least one fraction or combination of fractions to an alkaline pH enhances the thickening activity of said fraction. In an embodiment, the emulsification properties of the processed light fraction and/or the heavy fraction are believed to be improved when the pH is adjusted to be alkaline compared to fractions that have not been pH adjusted. In a preferred embodiment the pH is adjusted to be in the range of 7-11, more preferably 7.5-8.5. Particularly the processed light fraction and the heavy fraction exhibit a unique viscous behaviour as function of temperature and concentration. This property can be used in the preparation of emulsions and food systems. In particular, as the heavy fraction has a significantly larger viscosity compared to the processed light fraction, relatively low concentrations can be used to mimic food products with high fat contents. Blends of the fractions can be prepared to target specific food compositions and textures, such as yogurt, mayonnaise and tofu. Hence, the method may further comprise combining a portion of the heavy fraction with a portion of the light fraction or of the processed light fraction.

In a further embodiment, the light fraction which may have been concentrated and/or purified as described herein may be additionally processed by the steps of:
i. Adjusting the DW content of the processed light fraction to be 5-15%, preferably 10-15%;
ii. Subjecting the product of step i) to solid liquid separation, to obtain a mostly hydrophobic phase and a mostly hydrophilic phase;
iii. Collecting the hydrophilic phase obtained from step ii) (optionally subjecting said hydrophilic phase to mild drying, and/or optionally resuspending the resulting powder in water to reach a concentration of 0.1-200 g/L), and adjusting the pH of the collected hydrophilic phase to be in the range of 2-5, or in the range of 3.5-4.5, or preferably in the range 4-5.

This additional processing has been observed to improve the foaming properties of the (processed) light fraction, in that the resulting product has remarkable foamability and foam stability, in addition to improved heat-set gelation ability, making it suitable for food preparations that were not possible without the present method. Examples of such food preparations include cakes and confectionary products where both foaming and gelation ability are essential.

Solid-liquid separation in step ii) yields two phases; the top phase contains mostly hydrophobic compounds and the bottom phase contains mostly hydrophilic compounds. The top (hydrophobic) phase may have a whiteish colour, and/or may have a creamy texture to the touch.

In a preferred embodiment solid-liquid separation in step ii) is performed using a centrifugal separator. Examples of centrifugal separators include but are not limited to a cream separator, a bactofuge, and a bench centrifuge. The skilled person is able to select the right equipment and process conditions in order to obtain two phases, where the top phase can be skimmed-off and separated from the bottom phase.

In a further embodiment the processing step d) comprises preparing an aqueous suspension comprising the heavy fraction at 1-15% DW, preferably 5-15%, more preferably 10-15% DW and adjusting the pH of said heavy fraction aqueous suspension to pH 2-5, more preferably 4-5. This is observed to improve foaming properties including foam ability and/or foam stability. This outcome is particularly unexpected, as the skilled person would expect that the heavy fraction would have poor foaming functionality as the heavy fraction is comprised of mostly cell fragments with a size >1 μm, with a D50>0.5 (or in some embodiments, >4.0). This processed heavy fraction leads to the production of stable foams, and is particularly useful in food preparations as a partial replacer or ingredients such as but not limited to xanthan gum and/or carrageenan. Alternatively, the processed heavy fraction can be used in food preparations as a texturizer, and/or as a fat replacer, for example, in meat analogues.

In a further embodiment of the invention, the processing step may comprise subjecting a fraction to a thermal treatment; this may be the heavy fraction, the light fraction, or (preferably) the processed light fraction which has previously been concentrated and/or purified as described herein; alternatively, this may be a combination of the light fraction, heavy fraction, and/or processed light fraction. The thermal treatment may include drying; and/or heating. Drying may include but is not limited to freeze drying and spray drying. Drying can be carried out to obtain a powder, preferably with a moisture content of <10%. Preferably the dried powder is subsequently exposed to a heat source under controlled conditions. Preferably said heat source has a temperature in the range of 40-80° C., more preferably in the range of 70-80° C. In a preferred embodiment the dried powder is subjected to said heat source for a time period longer than 5 days, more preferably >10 days, even more preferably >15 days. The resulting thermally treated product has unique functional properties; for example, an increased water holding capacity and/or increased heat-set gelation capacity compared to an untreated product. Preferably the water holding capacity of said product is increased by a factor of at least 5 and/or the heat-set gelation ability of said product is increased by a factor of at least 2. Further, gels obtained using the thermally treated product are improved over gels obtained using the untreated product; such improved gels are more porous, dry, still, and stiff in structure than gels obtained with the untreated product that are smoother, moister and weaker.

In another embodiment of the invention gel-like structures can be prepared from the processed light fraction by either of the following steps:
i. The processed light fraction is stirred in an aqueous suspension with a DW content >5% at an acidic pH, preferably in the range of 3-6, more preferably in the range of 3.5-4.5 until a first gel-like structure forms; or
ii. The processed light fraction is stirred in an aqueous suspension with a DW content >5% at an alkaline pH, preferably in the range of 8-11, more preferably in the range of 9-11 resulting in a stiff gel-like structure.

The aqueous suspension comprises the processed light fraction at >5% DW, more preferably >10% DW, most preferably >15%, and less than 25%, less than 20%.

Preferably the pH of the aqueous suspension in step i) is adjusted to a pH<5, more preferably <4. Preferably the pH in step ii) is adjusted to a pH>9, more preferably >11. The stiff gel-like structure so produced has an appearance that resembles gelatine.

In another embodiment of the invention the processed light fraction can be used as a glazing agent and/or film making agent and/or browning agent by subjecting the processed light fraction to the following steps:
i. Preparing an aqueous suspension of the processed light fraction where said processed light fraction is at >1% DW;
ii. Incorporating said aqueous suspension in a food product, topically and/or within said food product;
iii. Subjecting said food product to thermal processes.

In a further embodiment, the heavy fraction and/or light fraction may be subjected to extended cell disintegration under alkaline conditions as described in PCT/EP2021/075137 (WO2022/058287) and PCT/EP2023/056907. The surprising properties of the resulting heavy phase have been reported in PCT/EP2021/075137 (WO2022/058287) and PCT/EP2023/056907. However, the light phase displayed unexpected functional properties, when subjected to extended disintegration, including improved water holding capacity, gelation hardness, foaming stability and emulsion stability. This increased functionality is described in example 2.

Examples of suitable food products include but are not limited to baked goods such as bread and meat analogues. The thermal processes include but are not limited to baking and/or frying. The resulting food product may be given a coating with a waxy texture and/or appearance on the surface of a food product, indicating a glazing effect. Alternatively, or in addition, the food product may have a browning effect, possibly also with increased crunchiness. Both glazing and browning are highly desirable properties required in food products and that traditionally are obtained with protein from vegetable and animal origin.

The present invention describes a method for producing microbial cell extracts and further processing steps to enhance the functional properties of said microbial cell extracts. The methods described in the present invention result from unexpected discoveries that are particularly unique in that no bio-chemical functionalization is required as is commonly reported in the scientific literature.

Also provided by an aspect of the present invention is an oil: water emulsion comprising a fraction obtained by a process as described herein. The invention also provides a food ingredient for providing a desired functional property to a food product, the functional property being selected from gelation, foaming, glazing, browning, texture, emulsifier; the food ingredient comprising a fraction obtained by a process as described herein. Still further is provided a food product comprising such a food ingredient; and/or a food product comprising such a fraction. The food product may be selected from a meat analogue; a dairy analogue including cheese, yogurt, butter, or cream analogues; a tofu analogue; a tempeh analogue; a mayonnaise analogue.

Although primarily described herein with reference to preparations obtained from yeast cells, the invention is not limited to the same. Various other microorganisms can be used. In embodiments, the microbe may be selected from fungi, including yeast (preferably Saccharomyces sp, more preferably brewer's or baker's yeast, or Pichia sp); plants, in particular microalgae (including Tetraselmis sp or Chlorella sp, for example C. vulgaris); and cyanobacteria (including Arthrospira sp, preferably A. platensis). The microbe may also be selected from bacteria, for example Methylobacterium sp. or lactic acid bacteria.

### BRIEF DESCRIPTION OF THE FIGURES

FIG. 1—Particle size distribution and particle sizes after cell disintegration by bead milling

FIG. 2—Particle size distribution and particle sizes after solid-liquid separation (classification) of the disrupted microbial biomass. The red line shows the light phase and the green line shows the heavy phase.

FIG. 3—Gel hardness of gels prepared from the light phase concentrated with membranes with different cut-offs at several diafiltration ratios.

FIG. 4—Gelation performance of the concentrated light fraction as a wet and dry ingredient at different concentration

FIG. 5—Stability of the concentrated light fraction (wet, 17% DW) between 0-15 days.

FIG. 6—Gel hardness of the concentrated light fraction across a range of pH's

FIG. 7—Emulsion systems to enhance gelation performance of the concentrated light fraction

FIG. 8—Viscosity of the concentrated light phase in an emulsion system at different shear rates.

FIG. 9—Emulsion stability and emulsion textures achieved by compositions comprising the concentrated light phase at different pH values.

FIG. 10—Emulsion systems obtained with compositions comprising the concentrated light phase and heavy phase at a low oil content without heating.

FIG. 11—Emulsion systems obtained with compositions comprising the concentrated light phase and heavy phase at a high oil content with heating.

FIG. 12—Emulsion systems obtained with compositions comprising the concentrated light phase and heavy phase at a high oil content without heating.

FIG. 13—Gel like structures obtained from the concentrated light phase at alkaline pH (centre), acidic pH (right) and raw suspension (left). Conditions for gel making are <4.5 in the acidic range and >10 for the alkaline range.

FIG. 14—Glazing and browning of products coated with the concentrated light phase and heavy phase at different concentrations (5, 10, 20% DW), and subjected to 2 different heating regimes, 200° C. for 25 minutes and 35 minutes. Egg-white proteins and uncoated products were used as controls.

FIG. 15—Illustration of D10, D50, D90 for a bimodal distribution based on volume.

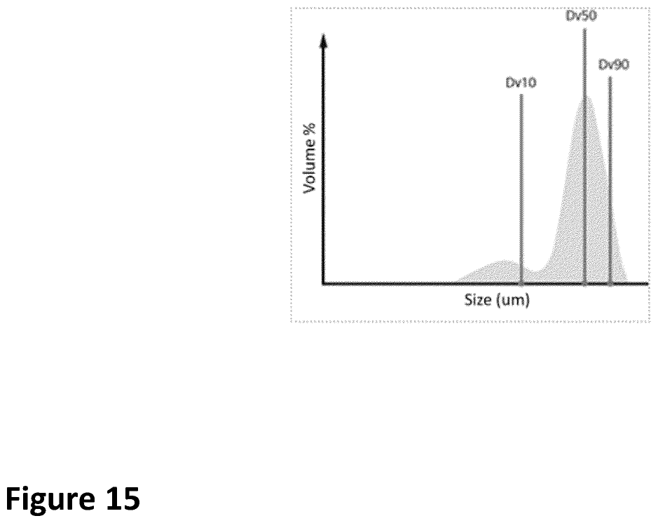

FIG. 16—Enrichment of particles from a disrupted suspension with a population of fragments (FIG. 16a) and separation into light and heavy phase under high centrifugal force (FIG. 16b), medium centrifugal force (FIG. 16c) and low centrifugal force (FIG. 16d). Phase split is indicated with a horizontal line.

FIG. 17—Psd of yeast biomass (circles), disrupted biomass (squares), the fraction enriched in small fragments (triangles) and the fraction enriched in large fragments (inverted triangles) according to the invention.

FIG. 18—Psd of the light phase after cell disintegration and centrifugal separation at several intensities (g forces): high intensity of 20000 xg for 15 minutes (triangles), medium intensity of 4000 xg for 15 minutes (squares) and low intensity of 1000 xg for 5 minutes (circles). The dashed square shows the desired range for particle enrichment.

FIG. 19—Gelation hardness for different recombination ratios of the light phase and the heavy phase according to the present invention.

FIG. 20—Psd of the disintegrated biomass, light phase and heavy phase derived from Methylobacterium spp. according to the present invention.

FIG. 21—Viscosities of emulsions prepared with several ratios of the light and heavy phase.

FIG. 22—Gelation hardness of gels prepared in emulsions and aqueous suspensions containing different weight ratios of the light (ME1) and heavy (ME2) phases (all samples are at a 10-12 wt % inclusion level).

### DETAILED DESCRIPTION OF THE INVENTION

The following embodiments apply to all aspects of the present invention.

The present invention will now be further described. In the following passages, different aspects of the invention are defined in more detail. Each aspect so defined may be combined with any other aspect or aspects or embodiment or embodiments unless clearly indicated to the contrary. In particular, any feature indicated as being preferred or advantageous may be combined with any other feature or features indicated as being preferred or advantageous.

The following definitions are used in the present description and claims to define the stated subject matter. Other terms not cited below are meant to have the generally accepted meaning in the field.

“Drying” as used in the present description means reducing the moisture content. The term drying includes partial drying wherein moisture may remain after drying in a reduced amount, which can also be seen as concentrating.

“Dry weight (DW)” and “dry cell weight” as used in the present description mean weight determined in the relative absence of water. For example, reference to microbial biomass as comprising a specified percentage of a particular component by dry weight means that the percentage is calculated based on the weight of the biomass after substantially all water has been removed.

“Disruption” as used in the present description in the context of microbial cells is also referred to as “lysing” and means opening the cells to release cytoplasmic compounds (also referred to as the “lysate”).

“Disintegration” as used in the present description means, in the context of disintegration of microbial cells, the fragmentation of the cells. This implies that the average size of the resulting cell fragments must be smaller than the average cell size of the initial microbial cells. Disintegration can be seen as a specific type of disrupting in which not only the cells are opened, but in which the cells are also fragmented.

“Cytoplasmic material” or “Cytoplasmic compounds” as used in the present invention means all material that is usually contained within a cell, enclosed by the cell membrane, except for the cell nucleus (if present). When a cell is disintegrated or disrupted, the cytoplasmic material is released from the cell.

“Microbial cells” as used in the present description means: microbes. This can be eukaryotic and prokaryotic unicellular organisms and colonies of them. A prokaryote is a cellular organism that lacks an envelope-enclosed nucleus. In the three-domain system, based upon molecular analysis, prokaryotes are divided into two domains: Bacteria (formerly Eubacteria) and Archaea (formerly Archaebacteria). Organisms with nuclei are placed in a third domain, Eukaryota. Microbial cells according to the present invention also encompass algae and fungi such as yeast.

“Microorganism” and “microbe” as used in the present description mean any microscopic colonial or unicellular organism.

“Microbial cell product” as used in the present description means: a product derived from microbial cells that is obtained by processing microbial cells in a certain manner.

“Light fraction” and “light phase” as used in the present description refers to the phase of a microbial cell extract enriched in small cell fragments (EESF) in the range of around 0.1-3 μm. “Small cell fragments” as used in the present description means cell fragments obtained from disintegration of microbial cells having a size of equal to or less than d50≤500 nanometers (nm). A light and a small fraction are used interchangeably herein.

“Heavy fraction” and “heavy phase” as used in the present description refers to the phase of the microbial cell extract enriched in large cell fragments (EELF) that are at a size of >1 μm. “Large cell fragments” as used in the present description means cell fragments obtained from disintegration of microbial cells having a size more than d50≥500 nanometer (nm). A heavy and a large fraction are used interchangeably herein.

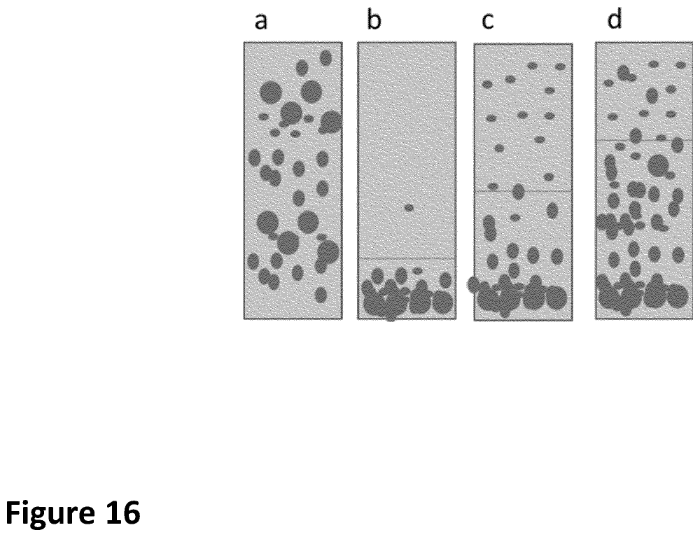

“Enriched” or “enrichment” as used in the present description means selective movement of particles to one of the two phases of separation i.e. the light phase or the heavy phase; this concept is illustrated in FIG. 16. When using high centrifugal forces, all particles/insoluble material are transferred to the heavy phase (FIG. 16b); if a centrifugal force is used that is too low the separation between the light and the heavy phase is poor and fragments are not clearly separated (FIG. 16d). However, using a mild or medium centrifugal force results in the small fragments being preferentially concentrated in the light phase (extract enriched in small fragments), while large fragments are preferentially concentrated in the heavy phase (extract enriched in large fragments), shown in FIG. 16c. The concept of enrichment is further described in example 14. Note that this is not the same as simply separating soluble and insoluble fractions, since insoluble material remains in both the light phase and heavy phase.

“Microbial biomass” and “biomass” as used in the present description mean a material produced by growth and/or propagation of microbial cells, or produced as byproduct of fermentation processes. Biomass may contain cells and/or intracellular contents as well as extracellular material. Extracellular material includes, but is not limited to, compounds secreted by a cell.

“Bead milling” as used in the present description means agitation of microbial cells in suspension with small abrasive particles (beads). Cells break because of shear forces, grinding between beads, and collisions with/between beads. Shear forces produced by the beads disrupt the cells and cause disintegration with concomitant release of cellular compounds.

“Centrifugation” as used in the present description means the application of centrifugal force to separate particles from a solution according to their size, shape, density, viscosity of the medium and rotor speed, among other parameters. The rate of centrifugation is specified by the angular velocity usually expressed as revolutions per minute (RPM), or acceleration expressed as g. The conversion factor between RPM and g depends on the radius of the centrifuge rotor. The general formula for calculating the revolutions per minute (RPM) of a centrifuge is

$$RPM = \sqrt{\frac{g}{r}}$$

where g represents the respective force of the centrifuge and r the radius from the center of the rotor to a point in the sample. However, depending on the centrifuge model used, the respective angle of the rotor and the radius may vary, thus the formula gets modified. The most common formula used for calculating Relative Centrifugal Force is:

$$RCF \text{(*} g ) = 1.118 * r * {( \frac{RPM}{1000} )}^{2}$$

wherein r is the radius in mm.

“Water holding capacity (WHC)” as used in the present description refers to the amount of water a sample can hold per unit of weight.

“Oil holding capacity (OHC)” as used in the present description refers to the amount of oil a sample can hold per unit of weight.

The present invention relates to a method for producing a microbial cell extract, methods of improving the functional properties of said microbial cell extract, and the use of said microbial cell extract in technical applications. In one embodiment the invention relates to the use of the microbial cell extract in a foaming agent, an emulsification agent, a thickening agent, a texturizing agent, a gelation agent or any other suitable application.

### EXAMPLES

The invention is described herein by the following non-limiting examples

### Example 1—Preparation of the Microbial Extracts

A microbial cell extract is prepared according to the process described in PCT/EP2021/075137. In summary a yeast suspension of the genus Saccharomyces, free of foreign contaminants, is subjected to bead milling under the following conditions:
a) : Biomass suspension at ˜100 g/L, pH adjusted from ˜4.5-5.5 to ˜9 using NaOH.
b) The suspension is bead milled at ˜20° C., 14 m/s tip speed, 75% bead filling, 0.5-1 mm zirconium beads, under batch recirculation mode.
c) The Temperature of the final disrupted suspension is ˜23° C., and the final pH is 5˜6.5.

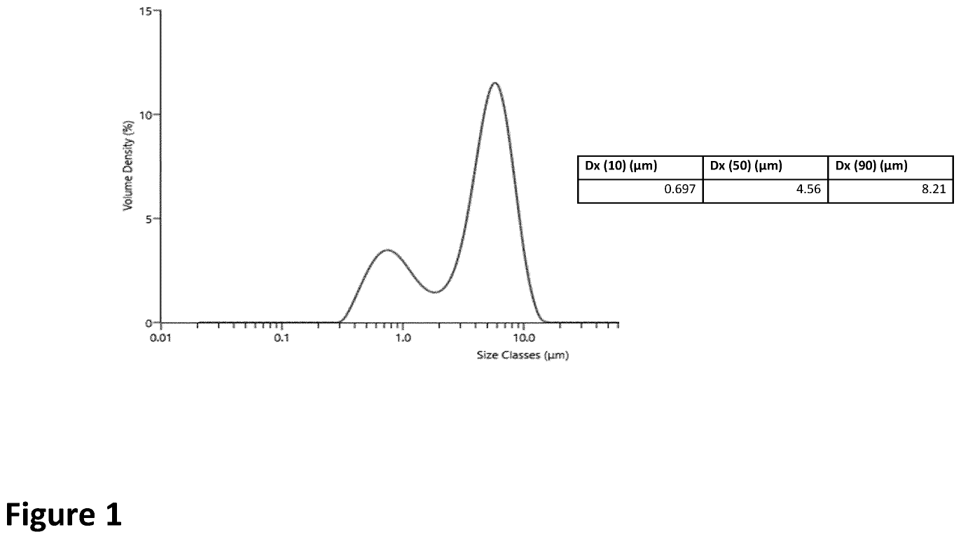

The particle size distribution (psd) and particle sizes following disintegration by bead milling are shown in FIG. 1.

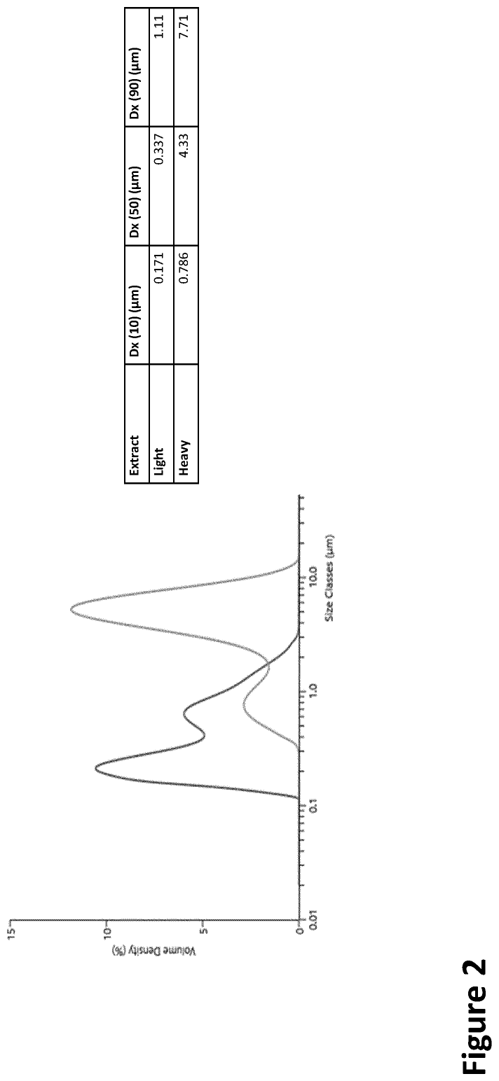

After disintegration, the disrupted microbial biomass was subjected to solid-liquid separation, also referred to as a classification step, to separate the disintegrated biomass into a light phase (also referred to as an extract rich in small fragments) and a heavy phase (also referred to as an extract rich in large fragments), wherein the extract rich in small fragments (light phase) consists of a population of soluble compounds and suspended fragments characterized by a bimodal distribution and a psd of D50 around 0.5 μm or lower; and the extract rich in large fragments (heavy phase) consists of a population of soluble compounds and suspended fragments having a psd of D50>4 μm. This separation was performed by centrifugation using a batch centrifuge, a 1 L volume at a speed 4000 xg for 15 minutes at 15° C. The particle size distribution (psd) and particle sizes of the light (red) and heavy phase (green) is shown in FIG. 2, and in table 1 below:

TABLE 1
Psd of the light and heavy phase

| Extract | Dx (10) (μm) | Dx (50) (μm) | Dx (90) (μm) |
|---|---|---|---|
| Light | 0.171 | 0.337 | 1.11 |
| Heavy | 0.786 | 4.33 | 7.71 |

The light phase or fraction obtained after classification by centrifugation is also referred to as a microbial cell extract enriched in small cell fragments. The heavy phase or fraction is also referred to as a microbial cell extract enriched in large cell fragments.

Both the light and heavy phase were then subjected to centrifugation using a bench centrifuge for 10 minutes at a 10 ml volume at as speed of 4000 xg for 15 minutes at ˜15° C. After this additional centrifugation step a new light and heavy phase were formed and the volumetric ratios were calculated as:

Light phase

$$Volume feed ( Vf ) - 10 ml$$

$$Volume heavy phase ( Vh ) - 0.5 ml$$

$$Volume light phase ( Vl ) - 9.5 ml$$

$$Volumetric ratio = \frac{0.5}{10} = 0.05$$

Heavy phase

$$Volume feed ( Vf ) - 10 ml$$

$$Volume heavy phase ( Vh ) - 9. ml$$

$$Volume light phase ( Vl ) - 1. ml$$

$$Volumetric ratio = \frac{9}{10} = 0.9$$

The resulting microbial cell extracts, namely the light and heavy phase were then further characterized. The composition of the light phase and heavy phase is shown in table 2 below, the composition of each extract was analysed with 5 replicates and the standard deviation was calculated.

TABLE 2
Mean composition (% DW) of the microbial extracts,
namely the light phase and heavy phase (n =
5 and SD = standard deviation)

| | Light | | Heavy | |
|---|---|---|---|---|
| Composition | Phase | SD | Phase | SD |
| Protein | 74.0 | 1.4 | 42.8 | 3.4 |
| Carbohydrates | 12.0 | 1.9 | 46.9 | 4.7 |
| Lipids | 5.4 | 2.2 | 1.8 | 0.5 |
| Ash | 5.6 | 0.6 | 9.5 | 5.7 |

### Example 2—Functionality of the Light Phase After Extended Bead Milling

This example describes the functional properties of the light phase derived from “extended disintegration” process reported in PCT/EP2021/075137 (WO2022/058287) and PCT/EP2023/056907. To the surprise of the inventors, the resulting light phase from said extended disintegration process also displays improved functional properties in comparison to those described for the reference process for the light phase (Ref, pH 9) (Table 3). As a reference, a process involving cell autolysis, cell homogenisation, alkaline extraction, acid extraction and aqueous extraction was compared to the current proposed method. The reference process has been reported by Saowanee Thammakiti, Manop Suphantharika, Thanaporn Phaesuwan, Cornel Verduyn. Preparation of spent brewer's yeast β-glucans for potential applications in the food industry. Food Science and Technology. Volume 39, Issue1, January 2004, Pages 21-29.

TABLE 3
Functional properties of the light phase prior to further processing.

| pH during | Gel hardness | WHC | OHC | Foam (½ | Emulsion |
|---|---|---|---|---|---|
| milling | [N] | [g/g] | [g/g] | time) [min] | stability |
| pH 11 | 0.3 | 3.2 | 1.3 | 15 | Very Stable |
| pH 9 | 0.1 | 2.6 | 1.0 | 3 | Stable |
| pH 4 | NA = No gel | 0.9 | 1.4 | 10 | Non-Stable |
| pH 2 | NA = No gel | 0.8 | 1.3 | 1 | Non-Stable |
| Ref, pH 9 | 0.21 | 1.1 | 1.1 | 2 | Stable |

### Example 3—Concentration of the Light Phase

The light phase was blended with distilled water at different ratios and the resulting suspension was subjected to filtration over a hydrophilic membrane PES with a cut off of 10 kDa and 100 kDa, keeping a transmembrane pressure <1 bar, at temperature of <25° C. The filtration process was conducted until a concentration factor 5x was obtained. For example, starting with 100 ml feed, the filtration was conducted until ˜20 ml retentate and ˜80 ml permeate was obtained. The filtration was conducted without pH adjustment. After filtration, the retentate fraction was collected for analysis of the heat-set gelation properties. This retentate is referred to herein as “concentrated light fraction” or light phase, or “processed light fraction” or light fraction; and may also be referred to in the examples and figures as “ME1s”.

The heat-set gelation properties are measured via double compression tests with a texture analyzer on samples at a constant DW content, after a heating treatment at 90° C. for 30 minutes followed by cooling to room temperature for 20 min.

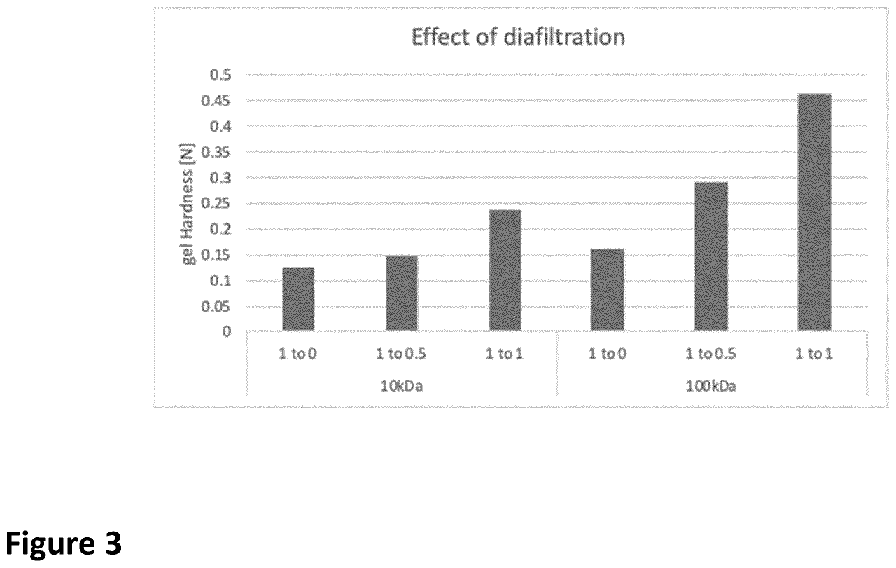

As presented in FIG. 3, the experimental data suggests that at a diafiltration ratio around 1:1 the gelation hardness, one of the most important parameters that determine heat-set gelation performance, was at its highest. It is also clear that a membrane of 100 kDa is optimal compared to a membrane of 10 kDa.

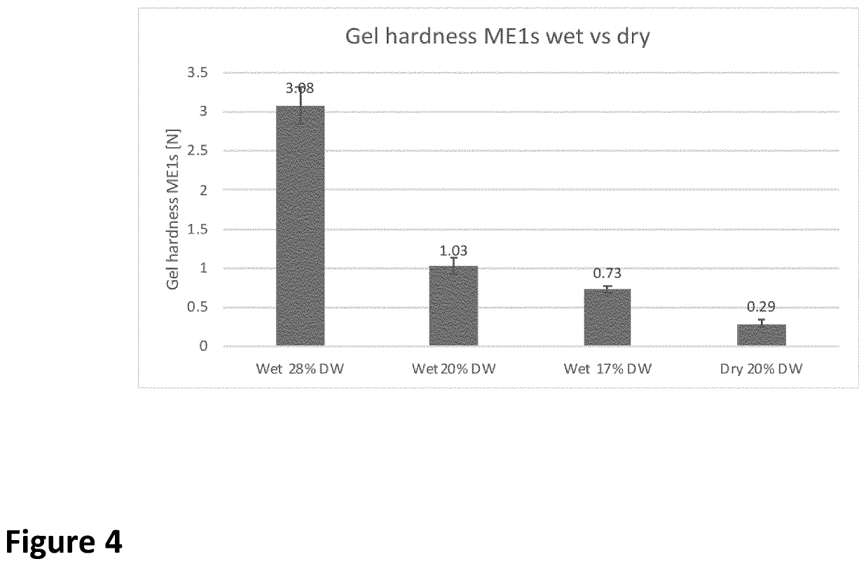

The gelation performance of the light fraction (measured as gelation hardness), obtained after concentrating the light fraction, is higher as wet ingredient compared to the dried and resuspended ingredient at the same DW content. As presented in FIG. 4, the gelation hardness at different concentrations does not follow a linear trend, but rather an exponential decay.

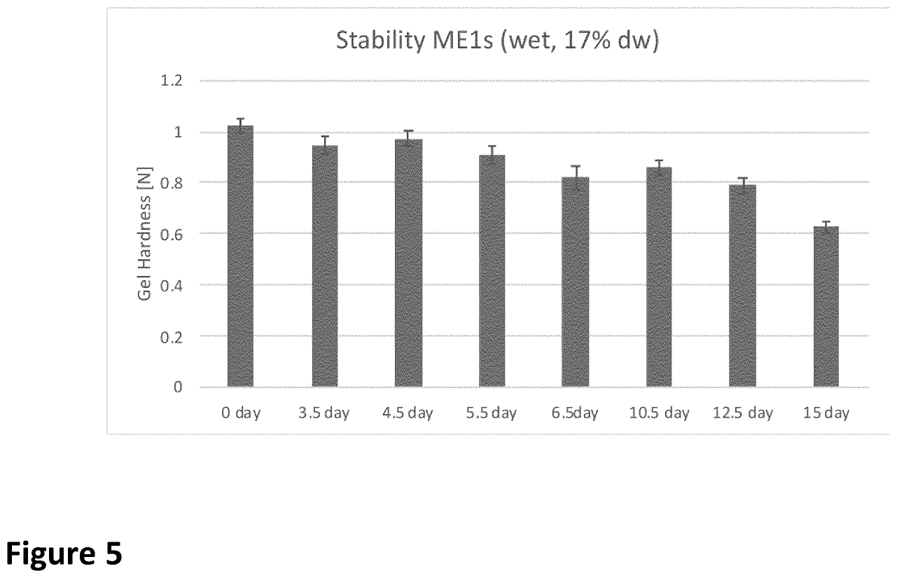

The stability of gelation performance of the concentrated light fraction (wet 17% DW) measured as gelation hardness was monitored over 15 days when stored in the dark and at ˜4° C., shown in FIG. 5, to assess whether the heat-set gelation properties of the light phase could be maintained for an extended period of time without the addition of stabilisers or preservatives. Here it can be seen that overall stability was high with gel hardness up to 5.5 days being very similar to what was observed before storage. Over the 15 days storage, gel hardness did not fall below 60% of what was achieved before storage, although after 5.5 days gelation hardness does begin to decrease steadily.

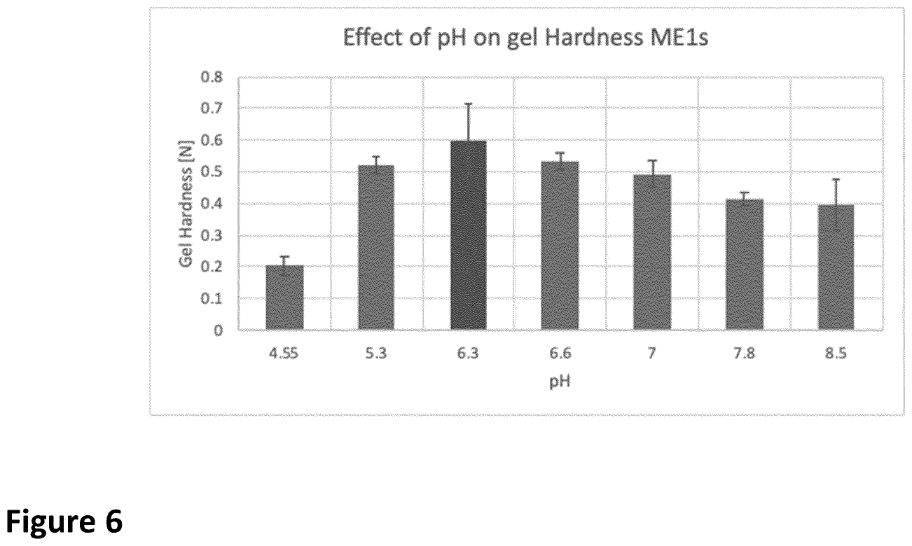

The effect of pH on the concentrated light fraction on gelation performance (measured as gelation hardness) was assessed over a broad range of pH's as shown in FIG. 6. Here is can be seen that gelation performance was optimal between pH 5.3-7.

### Example 4—Emulsion Systems to Enhance Gelation Performance of the Concentrated Light Phase

The concentrated light phase was prepared in different suspensions to evaluate its gelation properties. For every suspension tested the concentrated light phase was dried to form a powder and then dispersed in the relevant medium using a mid-shear stirrer so that in each suspension the light phase had a DW content 20%. Each suspension was then subjected to a standard heat gelation test and the gelation hardness of the resulting gel was measured using a texture analyser. The results obtained are shown below in table 4.

TABLE 4
Gels obtained from suspensions and emulsions
containing the light phase at DW 20%

| | | Gel hardness |
|---|---|---|
| Suspension | Observations | [N] |
| Aqueous | Good dispersibility, | 0.6 ± 0.1 |
| | slight foam formation | |
| Oil | Excellent dispersibility | No gelation |
| Emulsion W => O (the | Watery emulsion | 1.6 ± 0.2 |
| concentrated light phase | | |
| was first dispersed in | | |
| water, then the oil was | | |
| subsequently added | | |
| under shear to create an | | |
| emulsion) | | |
| Emulsion O => W (the | Creamy emulsion | 2.4 ± 0.1 |
| concentrated light phase | | |
| was first dispersed in oil | | |
| followed by the addition of | | |
| water to create an | | |
| emulsion) | | |

The results in table 4 show that there is a clear increase in the gelation hardness of the concentrated light phase in an emulsion system compared to when it is in only an aqueous suspension or only an oil suspension. Further to this, there is a clear advantage to preparing the emulsion by adding the concentrated light phase to the oil first before adding water as this achieves superior gelation hardness.

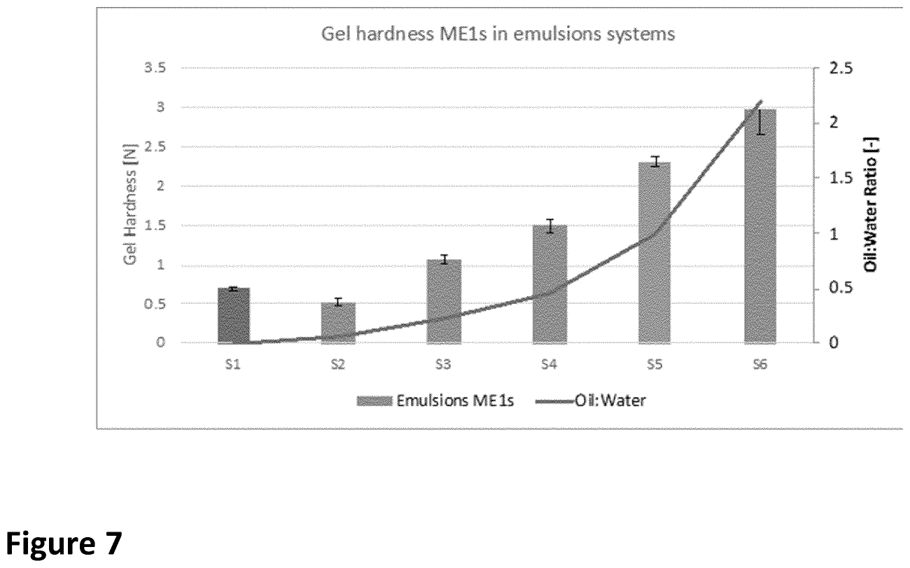

The gelation properties of emulsions comprising the concentrated light phase are also affected by the ratio of oil: water in the emulsion. The concentrated light phase was added to emulsions as 20% DW where said emulsions had ratios of oil: water ranging from 0-2.25, the gelation performance was measured by gelation hardness using a texture analyser. As shown in FIG. 7 the gelation hardness increases by almost 4 times when the ratio of oil:water is increased from 0 to ˜1. Beyond an oil:water ratio of 1 the gelation hardness increases even further, but the resulting gels at these higher ratios exhibit a darker colour, are more porous, brittle and have an uneven texture. In contrast the gels produced with an oil:water ratio of ˜1 are in comparison elastic, smooth, springy and appear light-beige in colour.

### Example 5—Unique Properties of Blends of the Light (ME1) and Heavy (ME2 Fractions

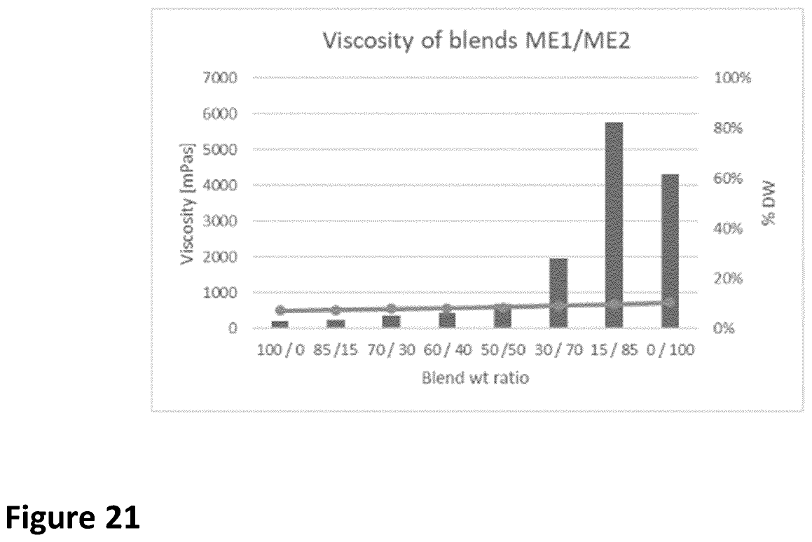

The example demonstrates the unique and unexpected performance of the microbial fractions obtained as described in the present invention. Powders obtained from the light and heavy fractions as described in example 1 after drying were incorporated into an emulsion using a high shear homogenizer (UltraTurrax, t50, IKA) at a ratio oil:water of 1:1 and a dry weight content of 5-7% (FIG. 21). The resulting emulsions were all stable (no phase split observed) for over 12 hours. As shown in FIG. 21, the viscosity of the resulting emulsions depended greatly on the ratio of the light fraction to the heavy fraction, even when the total dry weight content only varied in the range of 5-7%. Viscosities were measured at room temperature and a shear rate of 100 s⁻¹ using a dynamic rheometer MCR102 (AntonPaar).

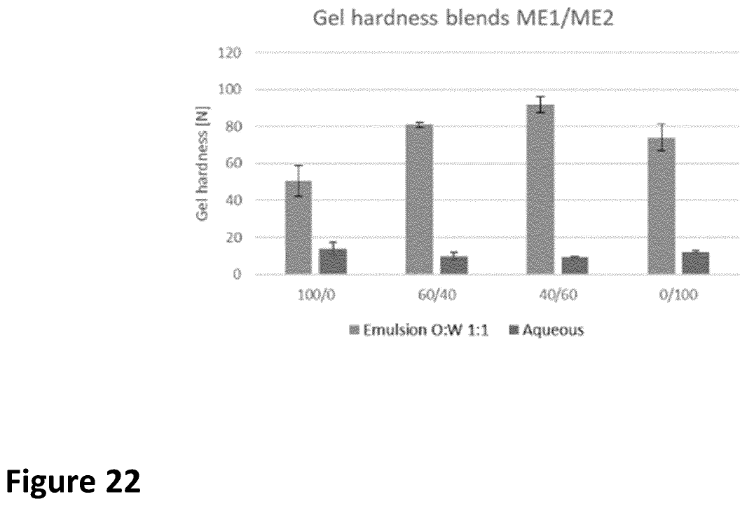

Furthermore, the emulsions were subjected to heating in a water bath at 90° C. for 30 minutes and then left to cool down to room temperature. The resulting gels were then analysed using a texture analyser (TA plus, Lloyd). The results in FIG. 22 show that, under an emulsion, the gelation properties of the microbial fractions are enhanced significantly and that there exists an optimal ratio of light fraction to heavy fraction which optimizes gelation hardness. Note that the optimal ratio for gelation is not the same as the ratio for maximum viscosity of the emulsion. This example clearly illustrates that the ratio of light phase to heavy phase can be used to achieve different textural properties.

### Example 6—Emulsion Performance of the Concentrated Light Phase and Heavy Phase

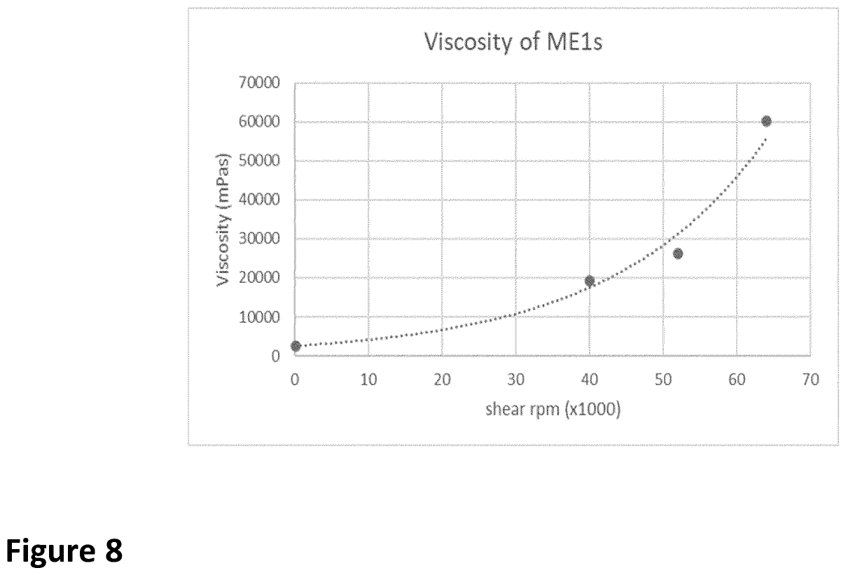

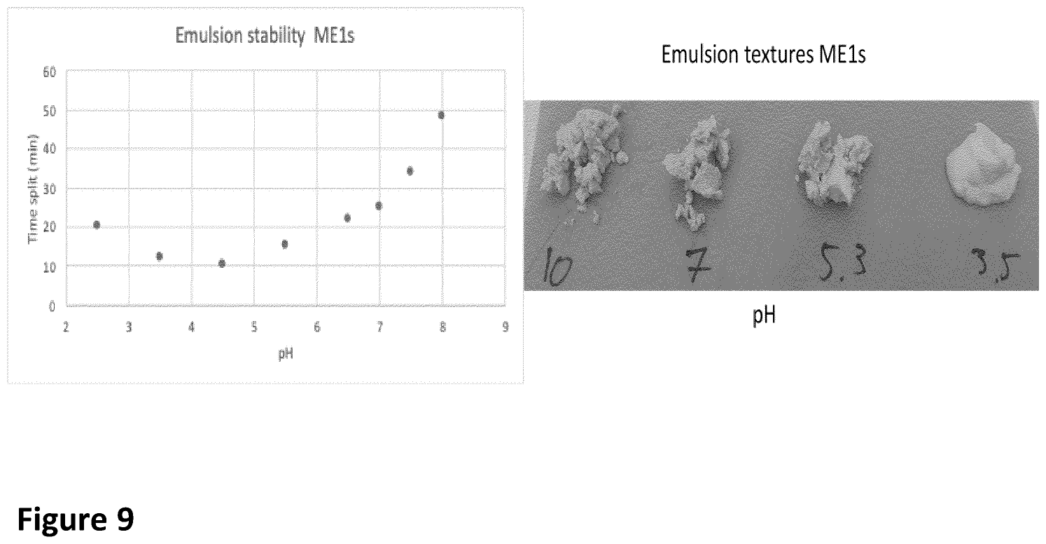

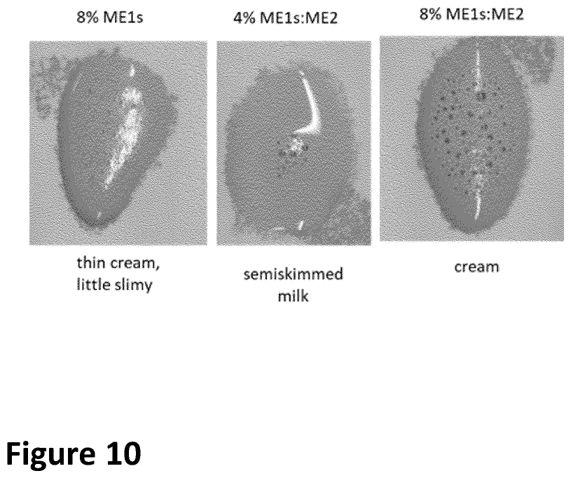

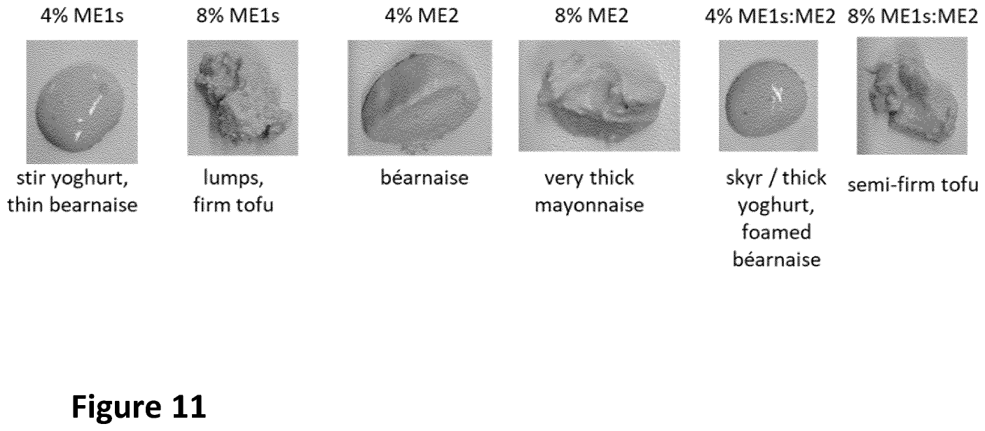

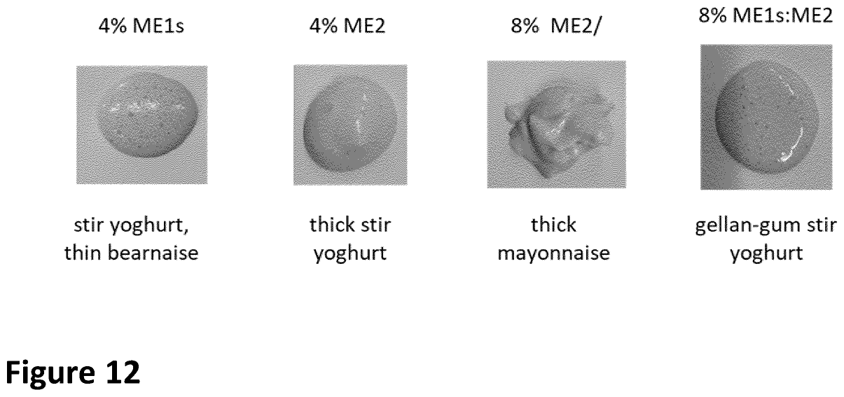

The emulsification behaviour of the concentrated light phase and heavy phase was investigated under several different conditions
1. Effect of Shear on Viscosity
Samples containing the concentrated light phase were prepared in an emulsion system containing 1:5.6:20 concentrated light phase: water: oil. The viscosity of this emulsion was then measured under a variety of shear treatments. Overall as strong correlation was observed between shear and viscosity (as shown in FIG. 8), demonstrating that the concentrated light extract behaves as a shear thickening material.
2. Effect of pH on Emulsions
The emulsification capacity and emulsion stability of emulsions prepared with the microbial extracts (concentrated light phase and/or heavy phase) can be improved by adjusting the pH of the suspensions they comprise. Experiments were conducted with concentrated light phase at 10% DW and pH values in the range 3.5 to 10. The resulting emulsion at pH 3.5 was soft and creamy, while the emulsion at pH 5.3, 7 and 10 was thick-creamy to sliceable. A thicker emulsion was produced at pH 10. In addition, the emulsion stability was evaluated at different pH levels. The stability was measured in terms of phase split (time until the emulsion phases start to separate). Emulsion stability and emulsion textures are shown in FIG. 9.
3. Effect of Processing Conditions on the Texture of Emulsions Comprising the Concentrated Light Phase and/or Heavy Phase.
Several prototypes were prepared comprising the concentrated light phase and heavy phase singularly and in combination, with variations in the oil content (high oil content=52%, and low oil content=13%), the content of microbial extract was tested at 4% and 8%, whilst the effect of heat treatment during emulsification was also assessed. (Note that “ME1s” refers to the concentrated light phase, while “ME2” refers to the heavy phase).
   At a low oil content (13%) without heating during emulsifications, textures varying from thin cream to skimmed milk and cream were obtained as shown in FIG. 10.
   At a high oil content (52%), with heating during emulsification, the resulting textures were more viscous and thicker. Textures comparable to yogurt, mayonnaise, and tofu were obtained. It also became evident that the contribution of the heavy phase (“ME2”) to the overall texture provides a more significant thickening behaviour in comparison to concentrated light phase (“ME1s”) as shown in FIG. 11.
   At high oil contents (52%), without heating during emulsification, several textures were obtained, such as stir yogurt and thick mayonnaise. Again, the thickening properties of the heavy phase were shown to play a key role in achieving the desired thick mayonnaise texture (FIG. 12). From these results it is clear that the heavy phase can be used as fat replacer in the production of food products such as mayonnaise (which normally requires at least 80% oil content).

### Example 7—Foaming Stability

Aqueous suspensions comprising the concentrated light phase and/or heavy phase can form foams when subjected to strong mechanical shear and/or bubbling. However, the foam stability is in general poor. It was found that the foam stability of the concentrated light phase and the heavy phase can be significantly extended by adjusting the pH of suspensions containing the concentrated light phase and/or heavy phase, as shown in table 5 below:

TABLE 5
Half times of foams produced comprising the concentrated
light phase (“ME1s”) and heavy phase (“ME2”) at
different pHs compared to an egg white control

Stability (½ times in h)

| | pH 6.5 | pH 4 |
|---|---|---|
| ME1s | 0.2 | >4.5 |
| ME2 | 0.1 | >4 |

| | |
|---|---|
| Egg white | >5 h |

To further improve the foam properties of the concentrated light phase, an aqueous suspension containing ˜15% DW of the concentrated light phase was subjected to centrifugation in a bench centrifuge at 3000 xg for 50 min at 15° C. After centrifugation, a two-phase system was formed where the top phase was ˜5% v/v while the bottom phase was ˜95% v/v. The top layer can be easily skimmed off using a mechanical element. The bottom phase was collected and the pH adjusted to ˜4.1, and the resulting suspension was used to prepare a meringue using standard recipes. As a control sample the concentrated light phase was used at the same DW content, but without pH adjustment or centrifugation step. During the whipping step, the adjusted concentrated light phase showed a volume increase of at least 500%, which remained stable. On the contrary, the concentrated light phase without pH adjustment and centrifugation, only showed a volume of increase of about 200%, which was unstable. During the baking step, the volume and structure of the meringue made with the adjusted concentrated light phase was comparable to meringues made with egg-white, while the meringues made with unadjusted concentrated light phase (without pH adjustment and centrifugation) were flat and soft.

### Example 8—Microbial Cell Extracts as Foam Stabilisers

Due to its properties as thickening agent, the heavy phase can be used as foam stabilizer. Experiments were conducted using a standard kitchen milk frother. Aqueous suspensions comprising the heavy phase were prepared at concentrations ranging from 1 to 10% DW, and subjected to 3 frothing cycles. The resulting foam was transferred to a volumetric cylinder where the foam stability, in terms of liquid drainage, was measured over time. Xanthan gum (XG) at 2% DW was used as a control. Foam ability is indirectly determined by measuring the weight of a 100 ml foam produced from the frother. Foaming ability and drainage (foam stability) results are shown below in table 6.

TABLE 6
Foaming ability (weight) and foam stability (weight)
for aqueous suspensions comprising the heavy phase
at 1-10% DW and a control Xanthan gum (XG)

| Sample | Weight (100 mL) | Drainage (ml/min) |
|---|---|---|
| Control | 35.7 | 0.56 |
| Heavy Phase (low) | 39.6 | 0.25 |
| Heavy Phase (medium) | 45.9 | 0.12 |
| Heavy Phase (high) | 61.2 | 0.00 |
| XG low | 37.1 | 0.26 |
| XG medium | 39.6 | 0.10 |
| XG high | 54.9 | 0.00 |

### Example 9—Foaming Properties of the Heavy Fraction (ME2)

The heavy fraction (ME2) was produced according to example 1 and subjected to mild drying, before being used in a model formulation to produce meringue like products. The ME2 was dispersed in water and the pH adjusted ˜4, followed by stirring and slow sugar addition until a glossy, foam like texture was obtained. A control experiment was also performed using egg white as a positive control. It is shown in table 7 that a unique sugar to ME2 ratio, even at lower inclusion levels, leads to the development of a foam-like structure with the desired properties of the positive control (c+).

TABLE 7
Foaming tests of the ME2
Foaming tests at several sugar/water/ME2 ratios (w/w)

| Recipe | Test 1 | test 2 | test 3 | C+ |
|---|---|---|---|---|
| Sugar | 66.7 | 66.7 | 33.35 | 66.7 |
| Water | 26.7 | 30.05 | 63.4 | 26.6 |
| ME2 | 6.7 | 3.35 | 3.35 | 0.0 |
| Egg White | 0.0 | 0.0 | 0.0 | 6.7 |
| total | 100.1 | 100.1 | 100.1 | 100 |

Foaming properties

| Property | Test 1 | Test 2 | Test 3 | C+ |
|---|---|---|---|---|
| Mass [g] | 397.3 | 383 | 398 | 398 |
| Volume [ml] | 450 | 460 | 855 | 780.2 |
| Density | 0.88 | 0.82 | 0.46 | 0.51 |
| [g/ml] | | | | |

### Example 10—Thermal Treatment of the Concentrated Light Phase

Powders of the concentrated light phase with ˜4.1% moisture, were placed in an aluminium tray and exposed to a thermal source at several temperatures. The treatments were conducted for 0 (untreated), 10 and 20 days. At the end of the treatment the powders were resuspended in water at 20% DW and measured to determine the water holding capacity (WHC) and the gelation performance using standard gel testing in a texture analyzer. The results in table 8 show that both the gelation hardness and water holding capacities increase significantly at high temperatures and exposure times. At 80° C. and 20 days, the gel structure is more porous, brittle and stiffer.

TABLE 8
The effect of thermal treatment on powders comprising
the concentrated light phase on gelation hardness
and water holding capacity (WHC)

| Sample | Gel hardness [N] | WHC |
|---|---|---|
| Untreated | 0.25 | 2.3 |
| 50° C., 10 day | 0.28 | 2.5 |
| 50° C., 20 day | 0.31 | 3.1 |
| 80° C., 10 day | 0.51 | 6.1 |
| 80° C., 20 day | 0.63 | 12.5 |

### Example 11—Gel Like Structures Comprising the Concentrated Light Phase

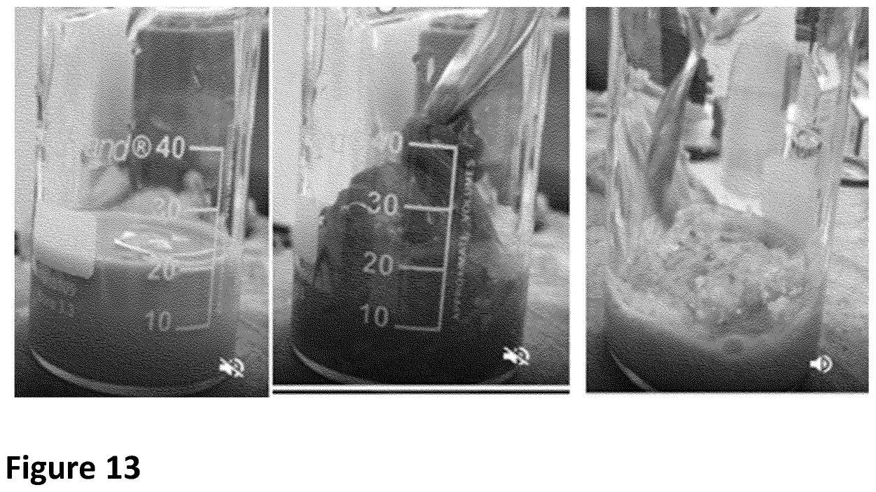

An aqueous suspension comprising ˜15% DW of the concentrated light phase was prepared and the pH of the suspension was adjusted using NaOH or HCl while under gentle mechanical stirring. After a gel-like texture was formed, the stirring was stopped (FIG. 13); the Figure shows gel like structures obtained at alkaline pH (center), acidic pH (Right) and raw suspension (Left). Conditions for gel making are <4.5 in the acidic range and >10 for the alkaline range.

### Example 12—Use of Microbial Cell Extracts as Glazing, Browning and Coating Agents

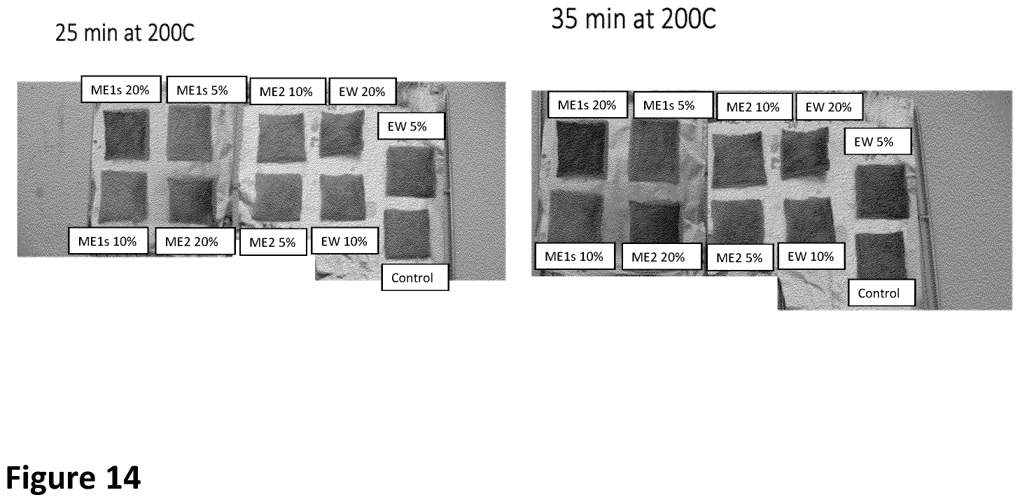

Aqueous suspensions comprising the concentrated light phase and/or heavy phase can be used as a glazing, browning and/or coating agent in diverse food preparations. Aqueous suspensions comprising the concentrated light phase and/or heavy phase in the range 5-20% DW were prepared and spread/coated on the surface a standard dough, followed by baking in oven at 200° C. for 25 minutes and 35 minutes. A coating of egg white and no coating at all were used as control samples. After baking, it was observed that the browning of the products comprising the microbial extracts was with the content of the concentrated light phase and heavy phase (higher concentrations result in darker colour). Colour intensities were comparable to the browning effect obtained from egg white and longer baking times resulted in a darker coloration (FIG. 14). In another example, an aqueous suspension comprising the concentrated light phase is coated over the surface of meat analogues and used as binder of bread crumbs for the preparation of chicken analogues.

### Example 13—Formulations and Food Products

This example shows several food products in which the microbial cell extracts and fractions described in the present invention deliver unique and surprising functionality.
a. Meat analogues

The light and heavy phase obtained according to the present invention can be used to replace binding agents such as hydrocolloids (e.g. methylcellulose) and proteins (e.g. egg white and potato protein) in burgers (vegetarian, raw, precooked) and other meat analogues (e.g. chicken and meatballs). Burgers prepared with the light and heavy phase (ME1 and ME2) after said light and heavy phase had been subjected to drying were rated as having comparable textural properties to the reference burger containing methylcellulose (table 9). Moreover, when the light and heavy phases are incorporated as an emulsion, the resulting burgers are juicier, firmer and present a better flavour profile.

TABLE 9
Burger recipes containing the light (ME1) and
heavy (ME2) fractions obtained according the
present invention and methylcellulose (MC).

| Burger recipe | MC | ME1 | ME1/ME2 |
|---|---|---|---|
| TVP | 23 | 23 | 23 |
| Water | 58.6 | 58.6 | 58.6 |
| oil | 8.2 | 8.2 | 8.2 |
| MC | 2 | 0 | 0 |
| ME1 | 0 | 3 | 2.8 |
| ME2 | 0 | 0 | 1.2 |
| Fiber | 2 | 2 | 2 |
| Other (flour, | 6.2 | 5.2 | 4.2 |
| herbs, flavors) | | | |
| total | 100 | 100 | 100 |

b. Sausages

The microbial cell extract light (ME1) and heavy (ME2) fractions can be incorporated into blends to create vegan sausages. Table 10 shows the qualitative assessment of sausages prepared with the light and heavy phase compared to egg white as a reference binding agent. This example illustrates that a blend of the dried light and heavy phase, incorporated as an emulsion, can be used to replace egg white to produce vegan sausages.

TABLE 10
Qualitative assessment of sausages prepared with the
light (ME1) and heavy (ME2) phase versus egg white.

| Binder | Qualitative assessment |
|---|---|
| 3% EggW | Dry and firm, good springiness |
| 3% ME1 | Dry, less firm, less springy |
| 3% ME1/ME2 40/60 | Juicy, firm, good springiness |

c. Pasta

The microbial cell extract light (ME1) and heavy (ME2) fractions have been used to replace egg whites in pasta.
d. Cheese analogues

The microbial cell extract light (ME1) and heavy (ME2) fractions have been used to prepare vegan cheese analogues. As shown in table 11, a cheese analogue was prepared containing the dried light (ME1) and heavy (ME2) fractions and compared to a cheese analogue containing potato protein. The resulting analogues showed comparable hardness as the reference, when the microbial cell extract is incorporated as an emulsion. Furthermore, the inclusion levels of the light (ME1) and heavy (ME2) phase and preparation method can be adjusted to prepare other types of cheese analogues, such as semi-hard, cream or cheese fillings.

TABLE 11
recipe for preparing a cheese analogue containing the light
(ME1) and heavy (ME2) phase and the corresponding hardness.

| | | | Hardness |
|---|---|---|---|
| Vegan cheese | amount | Condition | [N] |
| Water | 55.8 | No binding agent | 0.37 |
| Starch | 17 | ME1/ME2 | 0.49 |
| ME1/ME2 or Ref | 2 | ME1/ME2 in Emulsion | 1.5 |
| Salt | 0.2 | Ref: potato protein | 1.7 |
| oil | 25 | | |
| total | 100 | | |

e. Methylcellulose replacement

The microbial cell extract of the present invention can be used to replace methylcellulose and similar hydrocolloids (including HPMC/CMC) in potato products to preserve their shape during frying. The incorporation of the dried light (ME1) and/or heavy (ME2) phase (0.1-4% wt) in hot smashed potato leads to a stable product, with a better browning and crispiness compared to a reference sample containing 0.2-1% methylcellulose.
f. Other products

The microbial cell extract light and heavy fractions can be used to replace binding agents and emulsifying agents in a broad variety of formulations, food matrices and food products.

### Example 14—Enrichment

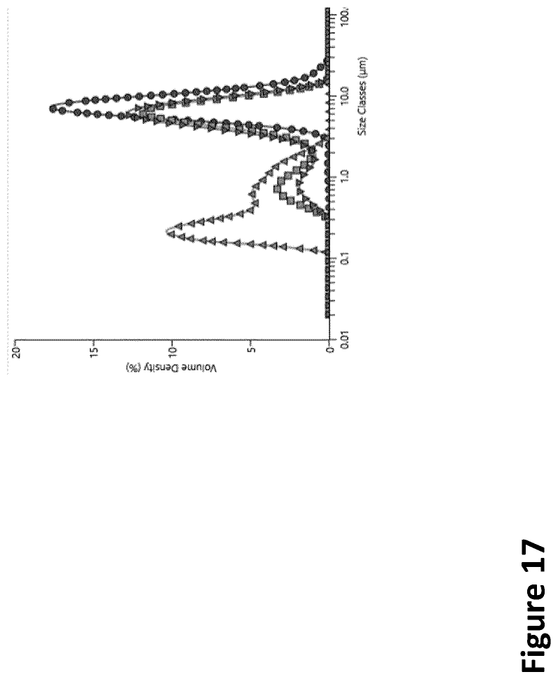

In an example demonstrating enrichment, a microbial biomass with a D50˜7.49 μm (depicted by the circles in FIG. 17) is disintegrated into a suspension with a bimodal distribution, represented by the squares in FIG. 17, with a D50 of ˜4.43 μm. The disintegrated biomass is subsequently separated into an extract enriched in small fragments, represented by the triangles with a D50˜0.35 μm, and a fraction enriched in large fragments, represented by the inverted triangles (FIG. 17) with a D50˜5.41 μm. Therefore, the light phase will be enriched in small fragments of sizes in the range 0.1-3 μm (D50<0.5 μm). Accordingly, the heavy phase will be enriched in large fragments of sizes >0.3 μm (D50>0.5 μm).

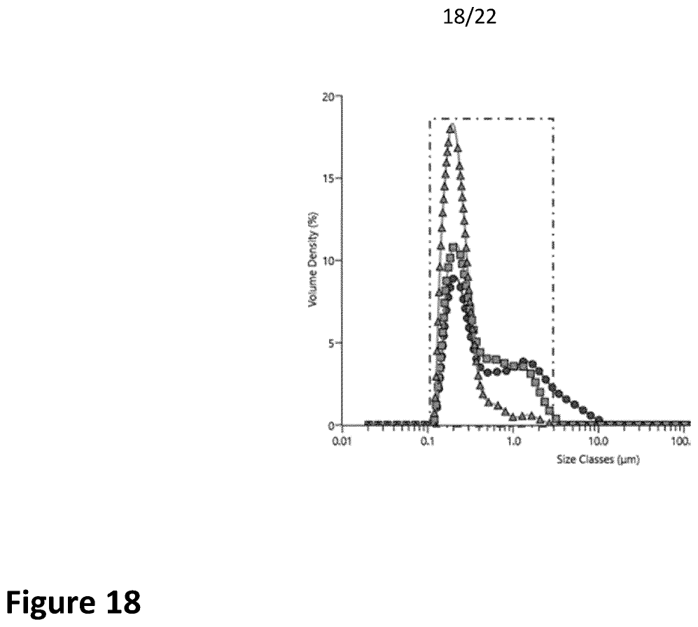

We have shown that there is a range of centrifugal forces that result in optimal functionality of the fraction enriched in small fragments. FIG. 18 shows a medium centrifugal force leads to superior enrichment of the fragments in the range of 0.1-3 μm, represented by the squares. Strong centrifugal forces (triangles) lead to a psd in the range of 0.1-0.4 μm, while low centrifugal forces result in a psd with fragments spanning to 10 μm (circles). The desired enrichment of small fragments is represented by the dashed square. If centrifugal forces are used which are too high or too low, this leads to a different psd and surprisingly worse functional performance, highlighting that obtaining a psd as defined by the present invention is critical.

Using different centrifugal forces affects the dry matter content of the light and heavy phase. Samples were produced according to the method of the invention as described above, but separated using mild and high centrifugal forces. Following separation these samples were subjected to the oven method known in the art, wherein the samples are kept at 100° C. in an oven until they are at a constant weight. The results of this are shown in table 12 below. A significant difference was observed in the dry matter content when different centrifugal forces were used. When using a mild centrifugal force of 4000 xg for 15 minutes there was additional dry matter in the light phase, due to there being more small particles that remain in suspension. This is an advantage of using a mild centrifugal force to achieve separation.

TABLE 12
Dry matter contents of the light and heavy phase
after centrifugal separation of disrupted biomass
using high and mild centrifugal forces.

| | Dry |
|---|---|
| | Weight |
| | % |
| High Centrifugal | |
| force: 20000 xg | |
| 15 min | |
| Light phase | 3.9% |
| Heavy phase | 17.2% |
| Mild Centrifugal | |
| force: 4000 xg, | |
| 15 min | |
| Light phase = FESF | 6.1% |
| Heavy phase FELF | 13.5% |

In previous publications that have reported producing microbial protein concentrates and protein isolates from a soluble fraction after cell lysis and centrifugation, centrifugal forces in the range of 10000-30000 xg are required to produce such soluble fractions and remove all insoluble compounds and particles. Traditionally, soluble fractions with a higher purity have been associated with high functionality and superior performance. However, unexpectedly the inventors have found that selectively enriching a fraction with small fragments resulted in superior functionalities as described above.

### Example 15—Functionality of the Recombined Light and Heavy Phase

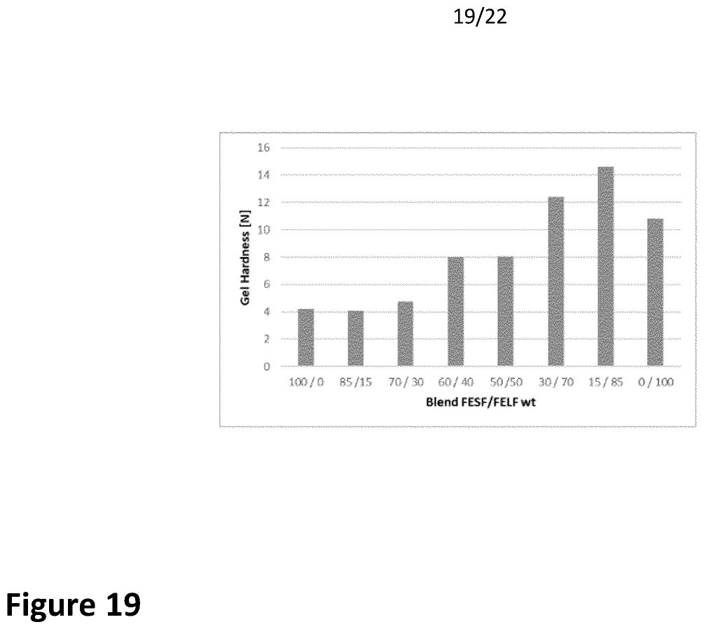

FIG. 19 shows several recombination ratios of the fraction enriched in small fragments and the fraction enriched in large fragments. The fractions were dried using spray drying recombined in a variety of ratios according to weight and assessed in terms of gelation hardness.

Gelation harness was measured after heat-set gelation in a water bath (15% DW suspension, heated at 90° C. for 30 min, followed by cooling at room temperature for 20 minutes and measuring hardness using a Texture analyzer Lloyd TA-Plus).

FIG. 19 shows optimal gelation hardness was achieved with a ratio of 15:85 of the EESF to the EELF. For reference, in this example the approximate ratio of both fractions prior to recombination was 40:60 which yielded a gelation hardness of ˜8 N.

It was unexpected that there was a ratio existing at which there was synergy between both fractions resulting in superior functionality, in this example, gelation hardness.

### Example 16—Functionality of Microbial Cell Extract Derived From Methylobacterium spp

A microbial cell extract was prepared according to the method described in PCT/EP2021/075137 (WO2022/058287). In summary, said microbial cell extract is produced by i) providing an aqueous suspension comprising microbial cells; ii) subjecting said suspension to mechanical cell disintegration, to obtain an aqueous suspension comprising disintegrated microbial cells; and iii) separating the suspension to provide an extract enriched in small cell fragments (“light phase”), and an extract enriched in large cell fragments (“heavy phase”). It is noted that optionally at least a portion of each extract may be recombined, to provide a recombined microbial cell product.

In this example the aqueous suspension comprising microbial cells comprises biomass of Methylobacterium spp at ˜100 g/L adjusted to ˜pH 9 with NaOH and subjected to cell disintegration via bead milling using 0.3 mm Zirconium beads, with a 65% filling rate, agitation speeds of 2039 rpm, and a temperature of ˜20° C.

The resulting disintegrated biomass particle size distribution is shown in FIG. 17, and represented by triangles, here it can be seen that a D50˜1.03 μm was achieved.

After the disintegration step, the resulting microbial suspension was subjected to centrifugation using a batch centrifuge at 4000 xg for 15 minutes at 15° C. This results in the formation of a light phase and a heavy phase of the microbial suspension (also referred to as an extract enriched in small cell fragments, and an extract enriched in large cell fragments, respectively).

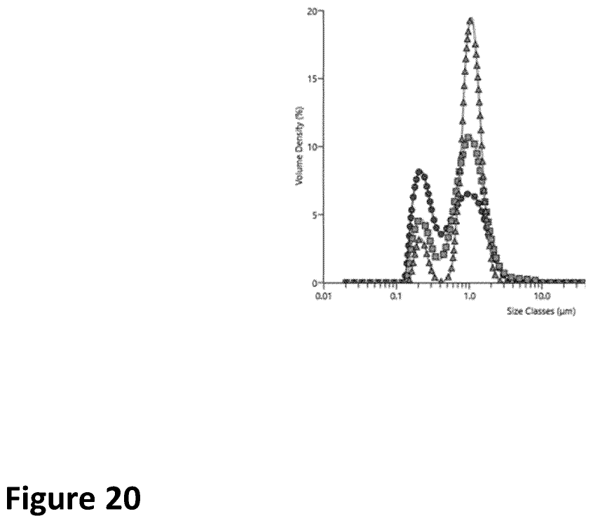

The particle size distribution and particle sizes of the light and heavy fractions resulting from separation by centrifugation are shown in FIG. 20 where the squares represent the heavy fraction and the circles represent the light fraction. The light fraction had a D50 of ˜0.57 μm. Meanwhile, the heavy fraction had a D50˜0.9 μm.

The resulting fractions were then analysed according to their functional properties as shown table 13 below. Here it is shown that the oil holding capacity and gelation performance were substantially improved in both the EESF and EELF compared to the disintegrated biomass.

TABLE 13
Functional properties of disintegrated Methylobacterium
spp and the resulting EESF and EELF

| Functional | Disintegrated | | |
|---|---|---|---|
| Property | Biomass | EESF | EELF |
| WHC [g/g] | 4.71 | 4.75 | 3.85 |
| OHC [g/g] | 2.23 | 3.15 | 4.61 |
| Gel | No, viscous | Yes | Yes |
| | paste | | |
| G′max [Pa] | 1.19 × 10⁵ | 1.17 × 10⁶ | 8.58 × 10⁵ |

Although primarily described herein with reference to preparations obtained from yeast cells, the invention is not limited to the same. Various other microorganisms can be used. In embodiments, the microbe may be selected from fungi, including yeast (preferably Saccharomyces sp, more preferably brewer's or baker's yeast, or Pichia sp); plants, in particular microalgae (including Tetraselmis sp or Chlorella sp, for example C. vulgaris); and cyanobacteria (including Arthrospira sp, preferably A. platensis). The microbe may also be selected from bacteria, for example Methylobacterium sp, or lactic acid bacteria.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

The scope of the present invention is defined by the appended claims. One or more of the objects of the invention are achieved by the appended claims

### REFERENCES

1. J. E. Kinsella, K. J. Shetty. Yeast Proteins: Recovery, Nutritional and Functional Properties. Nutritional Improvement of Food and Feed Proteins pp 797-825.
2. WO2020127951A2. FUNCTIONAL YEAST PROTEIN CONCENTRATE.
3. Vananuvat and Kinsella. Some Functional Properties of Protein Isolates from Yeast, Saccharomyces fragilis. J. Agric. Food Chem., Vol. 23, No. 4, 1975.
4. U.S. Pat. No. 3,888,839. Isolated yeast protein product with intact RNA and a process for making the same.
5. U.S. Pat. No. 3,887,431 Yeast protein isolate with reduced nucleic acid content and process of making same.
6. GB1578235A. Process for preparing functional yeast proteins using alkaline conditions
7. U.S. Pat. No. 5,756,135. Water insoluble yeast solids product and process of making same
8. U.S. Pat. No. 10,407,600B2. Yeast-based adhesive extract, composition and method
9. U.S. Pat. No. 2,603,630A. Method for extracting proteins from yeast
10. WO2006067145A1. New mannoprotein with full solubility in wine and its application in the stabilization of wine
11. WO2018002505A1 Use of a yeast protein extract to stabilize beer haze
12. Samara C. Silva, Isabel C. F. R. Ferreira, Madalena M. Dias, and M. Filomena Barreiro, Cristina González-Fernandez, Academic Editor. Microalgae-Derived Pigments: A 10-Year Bibliometric Review and Industry and Market Trend Analysis. Molecules. 2020 August; 25(15): 3406.
13. Gabriela Vollet Marson, Débora Tamires Vitor Pereira, Mariana Teixeira da Costa Machado, M. Di Luccio, Julian Martínez, M. Belleville, M. Hubinger. Ultrafiltration performance of spent brewer's yeast protein hydrolysate: Impact of pH and membrane material on fouling. 2021. Journal of Food Engineering 302(4):110569.
14. K J Shetty, J E Kinsella. Effect of thiol reagents on extractability of protein from yeast. Bioeng, Biotechnol 1978 May;20(5):755-66. R. Kollar, E. Sturdik, J. Sajbidor. Complete fractionation of saccharomyces cerevisiae biomass. Food Biotechnology. Volume 6, 1992—Issue 3.
15. J. A. Levesley, M. Seggiannl. M. Hoare. Microfiltration of Protein Precipitate from Clarified Yeast Cell Homogenate for the Recovery of a Soluble Product. Separation Science and Technology. Volume 35, 2000—Issue 5.

1. A method of preparing a microbial cell extract, the method comprising:
a) providing a microbial biomass in an aqueous alkaline suspension, at pH 7-11;
b) mechanically disintegrating the microbial biomass at a temperature below 40 deg C using a non-denaturing process, such that the disintegrated biomass consists of a population of soluble compounds and suspended fragments characterized by a bimodal distribution and a particle size distribution (psd) at an average of around D50<4.5 μm;
c) subjecting the disintegrated biomass to a solid-liquid separation process, to separate the disintegrated biomass into a light fraction (also referred to herein as an extract rich in small fragments) and a heavy fraction (also referred to herein as an extract rich in large fragments), wherein the light fraction consists of a population of soluble compounds and suspended fragments characterized by a bimodal distribution and a psd of D50 around 0.5 μm or lower; and the heavy fraction consists of a population of soluble compounds and suspended fragments having a psd of D50>0.5 μm; and
d) further processing either or both of the light fraction and heavy fraction to optimize one or more functional properties of said fraction(s), preferably for use in food manufacturing or cosmetics manufacturing.

2. The method of claim 1, wherein the microbial biomass is derived from yeast, more preferably from the genus Saccharomyces and/or Pichia.

3. The method of claim 1, wherein step d) comprises further processing the light fraction by purification and/or concentration.

4. (canceled)

5. The method of claim 3, wherein the light fraction is concentrated using dia-filtration where the ratio of water to the light fraction is approximately 1:1 and wherein the membrane used for diafiltration is in the range of 10 kDa-1000 kDa.

6. The method of claim 3, further comprising the step of drying the processed light fraction to remove moisture.

7. The method of claim 3, further comprising the step of adjusting the pH of the processed light fraction to the range 5-8.

8. The method of claim 1, further comprising combining two or more fractions selected from the light fraction, heavy fraction and processed heavy fraction.

9. The method of claim 1, wherein the processing step comprises preparing an emulsion of the fraction.

10. The method of claim 9 wherein the emulsion comprises a suspension of the fraction in a mixture oil:water at a mass ratio of between 0.25 to 2.5.

11. The method of claim 9, wherein the ratio of oil: water of the emulsion is between 0.25 to 1.5.

12. The method of claim 1, wherein the processing step comprises adjusting the pH of the fraction to an alkaline pH.

13. The method of claim 3, comprising additionally processing the processed light fraction by the steps of:
i. adjusting the DW content of the processed light fraction to be 5-15%, preferably 10-15%;
ii. subjecting the product of step i) to solid liquid separation, to obtain a mostly hydrophobic phase and a mostly hydrophilic phase;
iii. collecting the hydrophilic phase obtained from step ii) and adjusting the pH of the collected hydrophilic phase to be in the range of 2-5, preferably in the range 4-5.

14. The method of claim 1, wherein the processing step d) comprises preparing an aqueous suspension comprising the heavy fraction at 1-15% DW, and adjusting the pH of said heavy fraction aqueous suspension to pH 2-5.

15. The method of claim 1, wherein the processing step d) comprises subjecting a fraction to a thermal treatment.

16. (canceled)

17. The method of claim 15, further comprising combining two or more fractions prior to subjecting the combination to a thermal treatment.

18. The method of claim 3, comprising additionally processing the processed light fraction by the steps of either:
i. stirring the processed light fraction in an aqueous suspension with a DW content >5% at an acidic pH, until a first gel-like structure forms; or
ii. stirring the processed light fraction in an aqueous suspension with a DW content >5% at an alkaline pH resulting in a stiff gel-like structure.

19. The method of claim 3, comprising additionally processing the processed light fraction by the steps of:
i. Preparing an aqueous suspension of the processed light fraction where said processed light fraction is at >1% DW;
ii. Incorporating said aqueous suspension in a food product, topically and/or within said food product;
iii. Subjecting said food product to thermal processes.

20. The method of claim 1, further comprising incorporating the product of step d), or of any subsequent steps, into a food ingredient or a food product.

21. (canceled)

22. A food ingredient for providing a desired functional property to a food product, the functional property being selected from gelation, foaming, glazing, browning, texture, emulsifier; the food ingredient comprising a fraction obtained by a process as described in claim 1.

23. A food product comprising a food ingredient as recited in claim 22.

24. (canceled)
